(19) United States
(12) Patent Application Publication
(10) Pub. No.: US 2021/0107942 A1
(43) Pub. Date: Apr. 15, 2021

(54) TISSUE PROTECTIVE PEPTIDES AND PEPTIDE ANALOGS FOR PREVENTING AND TREATING DISEASES AND DISORDERS ASSOCIATED WITH TISSUE DAMAGE

(71) Applicant: Araim Pharmaceuticals, Inc., Tarrytown, NY (US)

(72) Inventors: Michael Brines, Woodbridge, CT (US); Anthony Cerami, Solana Beach, CA (US)

(21) Appl. No.: 16/878,318

(22) Filed: May 19, 2020

Related U.S. Application Data
(63) Continuation of application No. 14/905,754, filed on Jan. 15, 2016, now abandoned.
(60) Provisional application No. 61/847,455, filed on Jul. 17, 2013.

(51) Int. Cl. C07K 7/08; C07K 14/505; C07K 14/52; A61K 9/08

(52) U.S. Cl. CPC C07K 7/08; C07K 14/505; A61K 38/00; A61K 9/08; C07K 14/52

(57) ABSTRACT

The present invention provides peptides an peptide analogs that have tissue protective activities while having little or no potentionally undesirable hematopoietic effects. The peptides and peptide analogs are useful in preventing and treating a variety of diseases and disorders associated with tissue damage.

### CROSS REFERENCE TO RELATED APPLICATIONS

This application claims the benefit of priority of U.S. Provisional. Application Ser. No. 61/847,455, tiled Jul. 17, 2013, the entire contents of which are incorporated herein by reference.

### 1. INTRODUCTION

The invention provides tissue protective peptides and peptide analogs for preventing or treating a disease or disorder associated with tissue damage and/or damage, an effect, or a symptom thereof, including, but not limited to, cancer, inflammation, and exposure to a toxic agent. In particular, the invention provides tissue protective peptides and peptide analogs that share consensus sequences with fragments of Type 1 cytokine receptor ligands that have little or no potentially undesirable hematopoietic effects of the full length ligands.

These peptides also include fragments, chimeras, as well as peptides designed to mimic the spatial localization of key amino acid residues within the tissue protective receptor ligands, e.g., EPO. This invention further provides methods and uses of these peptides to modulate a subject's response and/or a symptom resulting from a disease or disorder associated with tissue damage for the purposes of treating, preventing or ameliorating the disease or disorder.

Additionally, the present invention provides pharmaceutical compositions comprising a peptide and a pharmaceutically acceptable carrier, excipient or diluent for the treatment of a disease or disorder associated with tissue damage; or damage, an effect or a symptom thereof, including, but not limited to, cancer, inflammation and exposure to a toxic agent, in a subject in need thereof.

### 2. BACKGROUND OF THE INVENTION

Tissue damage can be caused by a substantive loss of tissue due to ischemic, traumatic, toxic, or inflammatory injuries in which cells within the tissue are destroyed by apoptosis or necrosis. Tissue damage can occur in a number of acute and chronic, diseases and conditions. The degree to which tissue damaae occurs is mediated by many factors, including the type of disease or injury, the level of or severity of inflammation or trauma associated with the disease or injury, the location of the tissue damage, and the vascular sufficiency of the tissue.

Recent evidence suggests that erythropoietin (EPO), a member of the Type-1 cytokine family, commonly associated with the maintenance of hematocrit may also play an important role in attenuating tissue damage through the interaction with its receptor, EPOR (Brines et al,, 2004, Proc. Natl. Acad. Sci. USA, 101(41):14907-12). Although it is hypothesized that EPO may provide compensatory responses that serve to improve hypoxic cellular environment and modulate programmed cell death caused by metabolic stress, the underlying molecular mechanism is yet to be clearly understood.

Based upon this observation, investigators have explored the use of EPO various indications. As an example, investigators have explored the use of EPO as a potential treatment for cancers based upon the observation that EPO used to treat anemia in oncology patients not only rectified the anemia but resulted in an enhancement of the well-being of the oncology patient as well. (see U.S. Pat. No. 6,579,525 and Blau C. A., 2007, Stem Cells 25(8):2094-7). U.S. Pat. No. 6,579,525 to Haran-Ghera et al. relates to the use of recombinant EPO for the treatment of multiple myelomas and hypthesizes that EPO induces an immune response to the tumor, Additionally, U.S. patent application Ser. No. 11/093,177, publication no. US 2005/0267027, discloses the use of EPO to inhibit angiogenesis in tumors by reducing HIF-1α and/or VEGF expression in the tumors.

However, EPO as a potential tissue protective agent suffers from serious disadvantages due to its erythropoietic effect. In particular with chronic dosing, such as would be envisioned in indications such as cancer and inflammation, the frequent applications of therapeutic doses of EPO may significantly increase a subject's hematocrit, which may lead to hypertension, seizures, and vascular thrombosis.

Further, with regard to cancer, the potential of EPO as a therapeutic has not been realized. It has been determined that several types of cancer, such as breast cancers, express, and tend to over-express, erythropoietin receptors. This has led to concerns that the therapeutic use of EPO to treat cancer would lead to further growth of the tumor as opposed to a regression of the tumor's development (see Blau, 2007, supra, and U.S. patent application Ser. No. 10/432,899, published as US 200510260580). This concern has been borne out in the clinic as several trials of EPO within various cancer indications have been halted due to an increase in mortality due to tumor growth (Blau). In light of these adverse clinical outcomes the FDA has attached a Black Box warning on approved. EPO products cautioning against their use in unapproved cancer indications.

Additionally, mature human EPO protein is a 165 amino acid protein having a molecular weight of about 30.4 kDa measured by mass spectroscopy. The recombinant protein can be produced in Chinese hamster ovary cells in an expensive and labor intensive process that is highly regulated. Further, EPO must be stored under stringent conditions to maintain its activity. Given these limitations EPO is not an ideal candidate to address public emergencies, such as the release of a toxic agent such as radiation or a chemical agent, either through an industrial accident or act of terrorism or war that would require the rapid mass production of the therapeutic for wide distribution.

Accordingly, there is a need for tissue protective treatments that have little or no potentially detrimental effects and can be made readily available to the public.

### 3. SUMMARY

The present invention provides isolated peptides and peptide analogs that have tissue protective activity in a responsive cell, tissue, or organ. In certain embodiments, the peptides and peptide analogs have little or no potentially undesirable hematopoietic effects. In one embodiment, the tissue protective peptide has 15-29 amino acids and comprises the amino acid sequence of RYLLEAKEAENITFG (SEQ ID NO:1). In another embodiment, the tissue protective peptide consists of the amino acid sequence of RYLLEAKEAENITTG (SEQ ID NO:1).

In certain aspects, the invention provides isolated peptides and peptide analogs that have at least one tissue protective activity. Exemplary tissue protective activities include, but are not limited to, protecting, maintaining, enhancing, and restoring the function or viability of a responsive mammalian cell, tissue, or organ. Accordingly, in one aspect, the present invention provides the use of an isolated peptides and peptide analogs of the present invention for the preparation of pharmaceutical compositions for protecting, maintaining, enhancing, or restoring the function or viability of responsive mammalian cells and their associated cells, tissues, and organs. In related embodiments, the compositions are for administration to a subject in need thereof.

In other aspects, the isolated peptides and peptide analogs of the invention also have little or no erythropoietic activity, e.g. they do not significantly increase hemoglobin or hematocrit in a subject, or more generally have little or no hematopoietic activity, e.g. they do not significantly increase blood cellular components such as erythroid, lymphoid, and mycloid cells. In specific embodiments, the isolated peptides and peptide analogs have little or no activity selected from vasoactive action (e.g., vasoconstriction), hyperactivating platelets, prow coagulant activities, and stimulating proliferation or-production of thrombocytes or Serythropoietic-dependent cells (see, Coleman et 2006, Proc. Natl. Acad. Sci. USA 103.5965-5970).

The invention also provides pharmaceutical compositions comprising such tissue protective peptides and peptide analogs and a pharmaceutically acceptable carrier excipient or diluent, as well as methods for preparing such compositions and their use to treat diseases and disorders associated with tissue damage. In other aspects, the present invention provides methods of using an isolated peptide or peptide analog described herein for the preparation of a pharmaceutical composition for the protection against or prevention of a responsive tissue injury, for the restoration of, or for the rejuvenation of responsive tissue or responsive tissue function in a subject in need thereof. In one particular aspect, the responsive mammalian cells and their associated cells, tissues, or organs are distal to the vasculature by virtue of a tight endothelial cell barrier. In another particular aspect, the cells, tissues, organs or other bodily parts are isolated from a mammalian body, such as those intended for transplant. In certain aspects of the invention, the excitable tissue is central nervous system tissue, peripheral nervous system tissue, cardiac tissue or retinal tissue, In another aspect, the responsive cell or its associated cells, tissues, or organs are not excitable cells, tissues, or organs, nor do they predominantly comprise excitable cells or tissues.

In another embodiment the invention is drawn to a method of preventing, treating, ameliorating or managing inflammation, cancer or neoplastic disorders, or exposure to a toxic agent in a patient in need thereof by administering an effective amount of a peptide.

In certain embodiments, the invention relates to methods of modulating the activity of a mediator of cancer, the body's response to toxic agents, and inflammation. In particular, the invention relates to modulating the activity of an inflammatory mediator. Preferably, the peptides of the current invention are capable of modulating the effects of one or more inflammatory mediators.

In another embodiment, the invention relates to methods of arresting the growth of a cell comprising contacting a cell in need of growth arrestment with an effective amount of a peptide.

In another embodiment, the invention relates to methods of causing the death of a cancer or neoplastic cell comprising contacting a cancer or neoplastic cell with an effective amount of a peptide.

In another embodiment, the invention relates to methods of inhibiting blood vessel generation to the cancerous or neoplastic cells or reducing the production of molecules causing mitosis or angiogenesis.

In another embodiment, the invention relates to methods for treating or preventing the side-effects associated with chemotherapy or radiation therapy, comprising administering to a patient in need of such treatment or prevention an effective amount of a peptide. Side-effects associated with chemotherapy or radiation therapy include cachexia, low blood count, nausea, diarrhea, oral lesions, and alopecia.

In another embodiment, the invention relates to methods for treating or preventing cancer or neoplastic disease in a patient comprising contacting a cancer or neoplastic cell with an effective amount of a peptide.

In another embodiment, the invention relates to methods of treating or preventing cancer or neoplastic disease in a patient comprising administering to a patient in need of such treatment or prevention an effective amount of a peptide.

In certain embodiments, the invention relates to the use of the peptide for the preparation of a pharmaceutical composition for the prevention, treatment, amelioration, or management of cancer or neoplastic disorder in a subject in need thereof.

In another embodiment, the invention relates to methods of treating or preventing the symptoms associated with inflammation or an inflammatory condition. In a further embodiment, the invention relates to methods of treating or preventing inflammation or an inflammatory condition in a patient in need thereof. Amongst the inflammatory conditions treatable by the current method are allergies and allergic diseases, rheumatic diseases, andsports related injuries.

In another embodiment, the invention relates to method of treating, preventing, ameliorating or managing the effects of exposure to a toxic agent in a person in need of treatment. Amongst the toxic agents considered are biological, chemical and raidioactive agents.

In certain embodiments, the invention is also directed to pharmaceutical compositions comprising, the aforementioned isolated peptides for administration to a subject in need thereof. in specific aspects in accordance with this embodiment, the pharmaceutical composition of the invention further comprises a pharmaceutically acceptable carrier. Such pharmaceutical compositions may be formulated for oral, intranasal, ocular, inhalational, transdermal, rectal, sublingual, vaginal, or parenteral administration, or in the form of a perfusate solution for maintaining the viability of cells, tissues, or organs ex vivo. In related. embodiments of the invention the subject is a mammalian animal, preferably a human.

These and other features, aspects, and advantages of the present invention will become better understood with reference to the fallowing description and appended claims,

### 4. ABREVIATIONS AND TERMINOLOGY

4.1 Abbreviations

As used herein, the abbreviations for the genetically encoded enantiomeric amino acids are conventional and are as follows:

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| Pyroglutamate | U | pGlu (Glp) |

4.2 Terminology.

Unless defined otherwise, all technical and scientific terms used herein have the meaning commonly understood by a person skilled in the art to which this invention belongs. As used herein, the following terms have the meanings ascribed to them unless specified otherwise.

(i) As used herein, the terms “about” or “approximately” when used in conjunction with a number refer to any number within 1, 5, 10, 15 or 20% of the referenced number.

(ii) The term “administered in conjunction with” in the context. of the methods of the invention means administering a compound prior to, at the same time as, and/or subsequent to the onset of a disease, disorder, or condition.

(iii) The term “allergen” refers to an antigenic substance capable of producing immediate type hypersensitivity (allergy). Common allergens include, but are not limited to bacteria, viruses, animal parasites, insects and insect stings, chemicals (latex), dust, dust mites, molds, animal dander, drugs (such as antibiotics, serums, sulfa drugs, anti-convulsants, insulin preparations, local anesthetics, iodine, and aspirin), foods (such as milk, chocolate, strawberries, eggs, soy, nuts, fish, shellfish, wheat), perfumes, plants, pollens, and smoke.

(iv) The term “allergic disease” refers to a condition or disease caused by or relating to an allergy, Allergic diseases include, but are not limited to, asthma, hypersensitivity lung diseases, rhinitis, rhinosinusitis, atopic eczema, contact dermatitis, allergic conjunctivitis (intermittent and persistent), vernal conjunctivitis (hayfever), atopic keratoconjunctivitis, giant papillary conjunctivitis, urticaria (hives), angioedema, hypersensitivity pneumonitis, eosinophilic bronchitis, vasculitis, hypersensitivity vasculitis, antineutrophil cyte plasmic antibody (ANCA) associated vasculitis, Wegner's granulomatosis, Churg Strauss vasculitis, microscopic polyangiitis, temporal arteritis, celiac disease, mastocytosis, and anaphylaxis.

(v) The term “allergy symptom” or “allergic reaction” refers to the body's response to an allergen. The allergic reaction can be localized to one area (skin that came into contact with allergen) or generalized. Allergic reactions may include, hut arc not limited to, rash, itching, hives, swelling, difficulty breathing, wheezing, angioedema, difficulty swallowing, nasal congestion, runny nose, shortness of breath, nausea, stomach cramps, abdominal pain, vomiting and/or low blood pressure.

(vi) The term “allergy” refers to a state of hypersensitivity induced by exposure to a particular antigen (allergen) resulting in harmful immunological reactions on subsequent exposures.

(vii) The term “amino acid” or any reference to a specific amino acid is meant to include naturally occurring proteogenic amino acids as well as non-naturally occurring amino acids such as amino acid analogs. Those skilled in the art would know that this definition includes, unless otherwise specifically noted, naturally occurring proteogenic (L)-amino acids, their optical (D)-isomers, chemically modified amino acids, including amino acid analogs such as penicillamine (3-mercapto-D-valine), naturally occurring non-proteogenic amino acids such as norleucine and chemically synthesized amino acids that have properties known in the art to be characteristic of an amino acid. Additionally, the term “amino acid equivalent” refers to compounds that depart from the structure of the naturally occurring amino acids, but which have substantially the structure of an amino acid, such that they can he substituted within a peptide, which retains its biological activity despite the substitution. Thus, for example, amino acid equivalents can include amino acids having side chain modifications or substitutions, and also include related organic acids, amides or the like. The term “amino acid” is intended to include amino acid equivalents. The term “residues” refers both to amino acids and amino acid equivalents. Amino acids may also be classified into the following groups as is commonly known in the art: (1) acidic=Asp, Glu; (2) basic=Lys, Arg, His, (3) nonpolar (hydrophobic)=Cys, Ala, Val, Leu, Ile, Pro, Phe, Met, Trp, Gly, Tyr; and (4) uncharged polar=Asp, Gln, Ser, Thr. Non-polar may be subdivided into: strongly hydrophobic=Ala, Val, Leu, Ile, Met, Phe; and moderately, hydrophobic=Gly, Pro, Cys, Tyr, Trp. In alternative fashion, the amino acid repertoire can be grouped as (1) acidic=Asp, Glu; (2) basic=Lys, Arg, His, (3) aliphatic=Gly, Ala, Val, Leu, Ile, Ser, Thr, with Ser and Thr optionally be grouped separately as aliphatic-hydroxyl; (4) aromatic=Phe, Tyr, Trp; (5) amide Asp, Glu, and (6) sulfur-containing=Cys and Met. (Sec, for example, Biochemistry, 4th ed., Ed, by L. Stryer, WH Freeman and Co., 1995, which is incorporated by reference herein in its entirety).

(viii) The term “biological agent” as used herein refers to living organisms or the materials derived from them (such as bacteria, viruses, fungi, and toxins) that cause disease in or harm to humans, animals, or plants, or cause deterioration of materials. These biological agents are ubiquitous in nature and may be designed or optimized for use in warfare or terrorism. (bioterrorism). These biological agents may consist of prions, viruses, microorganisms (bacteria and fungi), and some unicellular and multicellular eukaryotes (i.e., parasites). In particular, the biological agents (identified by their common name, biologic name and the NATO Standard Reference letter code, where available) may include, but are not limited to, mycotic agents (Coccidioides mycosis, OC, Coccidioides posadasil, Coccidioides immitis), bacterial agents (anthrax (cutaneous, inhalation, gastrointestinal) (Bacillus anthracis, N and TR), plague (bubonic, pneumonic)(Yersinia pestis, LE), tularemia (Francisella tularensis, UL (schu S4), TT (wet type), ZZ (dry type) and SR and JT (425)), cholera (Vibrio choterae, HO), bovine brucellosis (AB), porcine brucellosis (US and NX), caprine brucellosis (AM and BX), Brucella abortus, Brucella melitenis, Brucella suis, bacterial dysentery (shigellosis, campylobacteriosis, salmonellosis) (Y), glanders (Burkholderia mallei, LA), melioidosis (Burkholderia pseudomallei, HI), diphtheria (Corynebacterium diphtheriae, DK), listeriosis (Listeria monocytogenes, TQ)), chlamydial agents (psittacosis “Parrot Fever” (Chlamydophilia psittici, SI), rickettsial agents (rocky mountain spotted fever (Rickettsia rickettsii, RI and UY), Q fever (Coxiella burnetti, OU, MN (wet type), and NT (dry type)), human typhus (Rickettsia prowazekii, YE), murine typhus (Rickettsia typhi, AV)), viral agents (yellow fever (Arbovirus flavivirdae, OJ, UT, and LU), rift valley fever (RVF Phlebovirus bunyaviridae, FA), alphaviruses (e.g.: eastern equine encephalitis (ZX), western equine encephalitis, Venezuelan equine encephalitis (NU, TD, and FX)), smallpox (ZL), Japanese B Encephalitis (AN), Cercopithecine herpesvirus 1 (Herpes B virus), Crimean-Congo haemorrhagic fever virus, viral hemorrhagic fever (Filoviridae (ebola and Marburg virus) and Arenaviridae (Lassa and Machupo)), monkey pox virus, Reconstructed 1918 influenza virus, South American Haemorrhagic Fever viruses (Flexal, Guanarito, Junin, Machupo, Sabia), tick borne meningoencephalitis (TEBV) viruses (Central European Tick-borne encephalitis, Far Eastern Tick-borne encephalitis, Kyasanur Forest disease, Omsk Hemorrhagic fever, Russian Spring and Summer virus), Hendra virus, Nipah virus, hantaviruses (Korean hemorrhagic fever), African horse sickness virus, optimized swine fever virus, Aka bane virus, avian influenza virus, bluetongue virus, camel pox virus, classical swine fever virus, foot-and-mouth disease virus, goat pox virus, lumpy skin disease virus, malignant catarrhal fever virus (Aleclaphine herpesvirus type 1), Menangle virus, New Castle disease virus, Pastes des petits ruminants virus, rabies virus, rinderpest virus, sheep pox virus, swine veseular disease virus, vesicular somatitis virus), toxins (botulinum toxin (Clostridium, X and XR), ricin (Ricinus communis, W and WA), Staphylococcal enterotoxin B (UC and PG), Saxitoxin (paralytic shellfish poisoning)(TZ and SS), tetrodotoxin (PP), conotoxins, Clostridium perfringens epsilon toxin, tricothecene mycotoxins (T-2 toxins), shigatoxin), and simuants (molasis residium (MR), Bacillus globigii (BG BS, and U), Serratia marescens (SM and P), Aspergillus fumigatus mutant C-2 (AF), E. Coli (EC), Bacillus thursidius (BT), Erwinia herbicola (EH), fluorescent particle (FP)), rye ergot, leprosy, rabies, intestinal typhoid, Clostridium perfringens (gas gangrene), aflatoxins, Salmonella typhimurium, enterotoxins, Argentinian hemorrhagic fever, multi-drug resistant Tuberculosis (MTB), Bolivian hemorrhagic fever, legionella pneumophilia, marine toxins, beriberi, malaria, pellagra, dengue fever, sclerotium rolfoil, neurotrophic encephalitis, Shigella (Y), SEB (UC), and mycotoxins, Diaacetoxyscirpenol, Cowdria ruminantium, Mycoplasma capricotum M.F38/M. Mycoides capri, Mycoplasma mycoides mycoides, abrin. The biological agents may be targeted. against humans (e.g. Small pox, Ebola virus, Reconstructed 1918 influenza virus, ricin, etc.), animals such as livestock (e.g. African horse sickness virus, African swine fever virus, foot-and-mouth disease, etc.) or both (Eastern equine encephalitis virus, etc.) Further, even non-lethal biological agents may pose a threat given that they may be re-engineered for greater lethality for use as a biological weapon. Thus even the viruses responsible for the common cold may pose a risk.

(ix) The term “cancer” as used. herein refers to any abnormal growth exhibiting malignant properties: the ability (1) to grow and divide without respect: to normal limits, (2) to invade and destroy adjacent tissues, and (3) in some instances, spread to other locations in the body. Cancer includes cancers or neoplastic disorders of the central nervous system, peripheral nervous system., gastrointestinal/digestive system, genitourinary system, gynecological, head and neck, hematological/blood, musculoskeletal/soft tissue, respiratory, and breast. Further examples of cancers or neoplastic disorders include, but are not limited to, those of the brain (astrocytoma, gliohastoma, glioma), spinal cord, pituitary gland, breast (infiltrating, Pre-invasive, inflammatory cancers, Paget's Disease, Metastatic and Recurrent Breast Cancer), blood (Hodgkin's Disease, Leukemia, Multiple Myeloma Lymphoma), Lymph node cancer, Lung (Adenocarcinoma Oat Cell, Non-small Cell, Small Cell, Squamous Cell, Mesothelioma), skin (melanoma, basal cell, squamous cell, Kapsosis Sarcoma), Bone Cancer (Ewings Sarcoma, Osteosarcoma, Chondrosarcoma), head and neck (laryngeal, pharyngeal (nasal cavity & sinus cavity), and esophageal cancers), oral (jaw, salivary gland, throat, thyroid, tongue, and tonsil cancers), eye, gynecological (Cervical, Endometrial, Fallopian, Ovarian, Uterine, Vaginal, and Vulvar), genitourinary (bladder, kidney, penile, prostate, testicular, and urinary cancers), adrenal (cortical adenoma, cortical carcinoma, pheochromocytoma) and gastrointestinal (appendix, bile duct (extrahepatic bile duct) colon, gallbladder, gastric, intestinal, colon, liver, pancreatic, rectal, and stomach cancers) as well as those listed below: (for a review of such disorders, see Fishman et al., 1985, Medicine, 2d Ed., J. B. Lippincott Co., Philadelphia): Leukemia: acute leukemia, acute lymphocytic leukemia, acute myelocytic leukemia, myeloblastic, promyelocytic, myelomonocytic, monocytic erythroleukemia, chronic leukemia, chronic inyelocytic (granulocytic) leukemia, chronic lymphocytic leukemia, Polycythemia vera, Gastric carcinoma, Lymphoma (malignant and non-malignant): Hodgkin's disease, non-Hodgkin's disease, Multiple myeloma, Waldenstroas macroglobutinemia, Heavy chain disease, Solid tumors sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, oral squamous cell carcinoma, hepatocellular carcinoma, basal cell carcinoma, adenocarcinoma, sweat gland carcinoma, sebaceous gland carcinoma, papillary carcinoma, papillary adenocarcinomas: cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, cervix adenocarcinoma, uterine cancer, testicular tumor, lung carcinoma, small cell lung carcinoma, non-small cell lung adenocarcinoma, bladder carcinoma, epithelial carcinoma, glioma, malignant glioma, glioblastoma, multiforme astrocytic gliomas, medulloblastoma, craniopharyngioma, ependymoma, pinealoma, hemangioblastoma, acoustic neuroma, oligodendroglioma, meningioma, melanoma, neuroblastoma, or retinoblastoma.

(x) The term “chemical agent” as used herein refers chemical substances that cause severe death or harm to people or animals. To the extent that the chemical agent has been optimized to be delivered using munitions or a dispersal device, the agent is a chemical weapon. In general, chemical agents used as weapons can be classified by their method of action such as: blood agents, blister agents, nerve agents, pulmonary agents, and incapacitating agents. Each of the chemical agents below are identified by their NATO standard Reference letter code where available.

(xi) “Blood agents” refer to those chemical agents that prevent cells from using oxygen. Chemical agents within this category include, but are not limited to Arsine (adamsite(diphenylaminechloroansinc), Clark I (diphenylchioroarsine), Clark II (diphenyleyanoarsine)) and Cyanide (cyanogen chloride (CK), hydrogen cyanide (AC), etc.) compounds. Arsine compounds cause intravascular hemolysis that leads to renal failure. Cyanide compounds prevent the cells from using oxygen and the cells then resort to anaerobic respiration creating an excess of lactic acid leading to metabolic acidosis. Victims of blood agents may exhibit symptoms including, but not limited to, headaches, dizziness, nausea, vomiting, mucosal irritation, dysponea, impaired consciousness, coma, convulsions, tachy- and brady-dysrhythmias, hypotension, cardiovascular collapse, and acyanosis.

(A) “Nerve agents” refer to those chemical agents that inactivate the enzyme acetylcholinesterase. The resulting buildup of the neurotransmitter acetylcholine in the victim's synapses leads to muscarinic and nicotinic effects. Compounds within this category include, but are not limited to, cyclosarin (cyclohexylmethylphosphofluoridate, GF), sarin (isopropyl mc.thylphosphanolltioridate, GB), thiosarin, soman (pinacolylmethylphosphanofluoridate, GC), tabun (ethyl N,N-dimethylphosphoramidocyanidate, GA), VX (O-ethyl-[s]-[2-diisopropylaminoethyl-methylphosphonothiolate), VR (N,N-diethyl-2-(methyl-(2-methylpropoxy)phosphoryl)sulfanylethanamine), VE (O-ethyl-S-(2-(diethylamino)ethyl]phosphonothioate), VG (O,O-diethyl-S-[2-(diethylamino)ethyl]phosphorothioate), VM (O-ethyl-S-[2-(diethylamino)ethyl]methylphosphonothioate), ethyl sarin (isopropylethylphosphonofluoridate, GE), EDMP (ethyl-2-diisopropylaminocthylmethylphosphonate), DF (methylphosphonyl difluoride), Novichok Agents, GV (P-[2-(dimethylamino)ethyl]-N,N-dimethylphosphonamidic fluoride), Gd42, Gd83, Tammelin Esters, fluorophosphocholines, phosphothiocholates, DFP, and insecticides (phenothiazines, organophosphates (dichorous, malathion, parathion, fenthion, amidon, paraoxon, chloropyritbs, systox, pyrophosphate, TOCP)). Victims of nerve agents may exhibit symptoms including, but not limited to, bradycardia, myosis, excessive salivation, vomiting, diarrhea, involuntary micturition, muscle fasciculation, initial depolarizing flaccid paralysis, spike discharges and convulsions, intermediate syndrome, neurotoxic esterase inhibition, and organophosphate-induced delayed neuropathy.

(B) “Blister Agents” refer to agents that are acid-forming compounds that damage the victim's skin and respiratory system resulting in burns and respiratory problems. Chemical agents within this category include, but arc not limited to, sulfur mustards (1,2 bis(2-chloroethylthio)ethane (Sesquimustard, Q), 1,3 bis(2-chloroethylthio)-n-propane, 1,4-bis(2-chloroethylthio)-n-butane, 1,5-bis(2-chlonoethylthio)-n-pentane, 2-Chloroethylchloromethylsulfide, Bis(2-chloroethyl)sulfide (HD), Bis(2-chloroethylthio) methane, Bis(2-chloroethylthiomethyl) other, Bis(2-chloroethylthioethyl) ether, di-2′-chloroethylsulfide and combinations (hereof (HT, HL, HQ)), nitrogen mustards (Bis(2-chloroethyl)ethylaminc (HN1), Bis(2-chloroethyl)methylamine (HN2), Tris(2-chloroethyl)amine (HN3), 2-chloro-N-(2-chloroethyl)-N-methylethanamine-N-oxide hydrochloride, cyclophosphamide, chlorambucil, uramustine, melphalan), lewisites (2-Chlorovinyldichloroarsine, Bis(2-chlorovinyl)chloroarsine, Tris(2-chlorovinyl)arsine, dichloro(2-chlorovinyl)arsine), ethyldichloroarsine, methyldichloroarsine, phenyldichloroarsine, and phosgene oxime (dichloroformoxime). Victims of blister agents may exhibit symptoms including, but not limited to, erythema, edema, necrosis and vesicles, melanoderma, tracheobronchitis, bronchospasms, bronchial obstruction, hemorrhagic pulmonary edema, respiratory failure, bacterial pneumonia, eye erythema, lachrymation, discomfort of the eyes, severe pain in the eyes, blepharospasm, iritis, blindness, nausea, vomiting, bone marrow suppression, lewisite shock, hepatic necrosis, and renul failure secondary to hypoperfusion.

(D) “Pulmonary Agents” refer to agents that arc similar to blister agents but have a more pronounced effect on the respiratory system resulting in the respiratory system being flooded and the victim suffocating. Chemical agents within this category include, but are not limited to, adamsite, Acrolein, Bis(chloromethyl)ether, chlorine, Chloropicrin, diphosphogene, methyl chlorosulfate, stannic chloride, hydrogen chloride, nitrogen oxides, and phosgene. Victims of pulmonary agents may exhibit symptoms including, but not limited to, burning sensations (eyes, nasopharynx, oropharynx), profuse tearing, rhinorrhoca, coughing hoarseness, dyspnoea, odynophagia, conjunctivitis, comeal injury, naso-orophangyal injury/edema, respiratory distress due to inflammation of the glottic structures, secretions, and/or laryngospasms, acute respiratory syndromes, and reactive airway dysfunction syndrome.

(E) “Incapacitating agents” refer to agents that are less lethal and are intended largely to incapacitate through physiological or mental effects or both. A common class of incapacitating agents is lachrymatory agents, chemical agents that irritate the eyes causing tearing, pain, and even temporary blindness. Lachrymatory agents include, but are not limited to a-chlorotoluene, benzyl bromide, Bromoacetone (BA), Bromobenzylcyanide (CA), Bromomethyl ethyl ketone, Capsaicin (OC), Chloracctophenone (CN), chloromethyl chloroformate, Dibenzoxazcpine (CR), Ethyl iodoacctate, Ortho-chlorobenzylidene malonitrile (CS), Trichloromethyl chloroformute, and xylyl bromide. Additional incapacitating agents include, but are not limited to, 3-Quinuclidinyl benzilate (psychedelic; BZ), hydrocyanic acid (paralytic), diphenyichloroarsine (sternutatory; DA), diphenylcyanoarsine (DC), KOLOKOL-1 (fentanyl derivative), Datura stramonium, Hellborne, Belladonna, Hyoscyamus falezlez, indoles (lysergic acid diethylamide (LSD-25)), marijuana derivatives (DMHP), amphetamines, cocaine, caffeine, nicotine, strychnine, metrazole, barbiturates (methohcxital), opioids, antipsychotics (halopcridol), benzodiazepines, fentanyl congeners, psilocybin, ibogaine, harmine, ectasy, PCP, atropine, scopolamine, oxybutynin, ditropan, anticholinergic antihistamine benactyzine, and tranquilizers.

Many of the above noted chemicals have uses beyond their use as weapons and are used within manufacturing. Thus, the accidental or intentional release of these chemical agents from manufacturing or chemical plants will pose a risk to the employees of the plant as well as the populations living in the vicinity of these plants. Examples of toxic industrial manufacturing chemicals include, but are not limited to, ammonia, arsine, boron trichloride, boron trifluoride, carbon disulfide, chlorine, diborane, ethylene oxide, fluorine, formaldehyde, hydrogen bromide, hydrogen chloride, hydrogen cyanide, hydrogen fluoride, hydrogen sulfide, nitric acid, phosgene, phosphorous trichloride, sulfur dioxide, sulfuric acid, tungsten hcxafluoride, acetone cyanohydrin, acrolein, acrylotrile, allyl alcohol, allyl amine, allyl chlorocarbonate, boron tribromide, carbon monoxide, carbonyl sulfide, chloroacetone, chloroacctylnitrile, chloro sulfonic acid, diketone, 1,2-dimethyl hydrazine, ethylene dibromide, hydrogen selenide, methane sulfonyl chloride, methyl bromide, methyl chloroformate, methyl chlorosilane, methyl hydrazine, methyl isocyanate, methyl mcrcapatan, nitrogen dioxide, phosphine, phosphorous oxychloride, phosphorous pentafluoride, selenium hexafluoride, silicone tetrafluoride, stiloine, sulfur trioxide, sulfuryl chloride, sulfuryl fluoride, tellurium hexafluoride, n-octyl mercaptan, titanium tetrachloride, trichloroacctyl chloride, trifluoroacetyl chloride, allyl isothiocyanate, arsenic trichloride, bromine, bromine chloride, bromine penta fluoride, bromine trifluoride, carbonyl fluoride, chlorine penta fluoride, chlorine trifluoride, chloroacetylaldehyde, chloroacetylchloride, crotonaldehyde, cyanogens chloride, dimethyl sulfate, diphenylmethane-4,4′-diisocyanate, ethyl chloroformate, ethyl chlorothioformate, ethyl phosphonothioic dichloride, ethyl phosphonic dichloride, ethyleneimine, hexachlorocyclopentadiene, hydrogen iodine, iron pentcarbonyl, isobutyl chloroformate, isopropyl chloroformate, isopropyl isocyanate, n-butyl chloroformate, n-butyl isocyanate, nitric oxide, n-propyl chloroformate, parathion, perchloromethyl mercaptan, sec-butyl isocyanate, tert-butyl isocyanate, tetraethyl lead, tetraethyl pyrophosphate, tetra methyl lead, toluene 2,4-diisocyanate, and toluene 2,6-diisocyanate.

(xi) As used herein, an “effective amount” includes that amount of a peptide sufficient to modulate any disease or disorder associated with tissue damage or the damage, effects, or symptoms thereof, preferably to inhibit, suppress, or moderate the deleterious effects of the body's response to the disease or disorder associated with the tissue damage including, but not limited to, the body's response to cancer, inflammation, or exposure to toxic agents. Additionally, an “effective amount” includes the amount of the peptide sufficient to mitigate, ameliorate, diminish or prevent any disease or disorder associated with tissue damage or provide a therapeutic benefit in a patient afflicted with a disease or disorder associated with tissue damage.

(xii) As used herein, “erythropoietic activity” means any significant increase in the levels of hemoglobin or hematocrit in a subject. “Little or no erythropoietic activity” means that an increased level of a subject's hemoglobin or hematocrit meets the criteria accepted in the art as an insufficient increase to cause art adverse effect in a subject. “Significantly increased erythropoietic activity” means that the a difference in the level of a subject's hemoglobin or hematocrit compared to a control meets the criteria accepted in the art as significant, which may, inter alia, increase the likelihood of hypertension, seizures, and vascular thrombosis.

(xiii) “Excitable tissue” means tissue that contains excitable cells. Excitable cells are cells that respond actively to an electric stimulus and have an electrical charge differential across their cellular membranes. Excitable cells are generally capable of undergoing an action potential. Such cells typically express channels, such as voltage-gated, ligand-gated, and stretch channels, which allow flow of ions (potassium, sodium, calcium, chloride, etc.) across the membrane. Excitable tissue includes neuronal tissue, muscle tissue, and glandular tissue. Excitable tissue includes, but is not limited to, neuronal tissues such as tissue of the peripheral nervous system (ear and retina) and central nervous system (brain and spinal cord); cardiovascular tissue such as the cells of the heart and associated nerves; and glandular tissue such as the pancreas where T-type calcium channels along with cell-to-cell gap junctions participate in secretion of insulin. An exemplary list of excitable tissue includes organs and tissues that include nerves, skeletal muscle, smooth muscle, cardiac muscle, uterus, central nervous system, spinal cord. brain, retina, olfactory system, auditory system, etc.

(xiv) The term “hematopoictic activity” means any significant increase in blood cellular components such as erythroid, lymphoid, and myeloid cells. Further hematopoietic activity refers to whether an isolated peptide or peptide analog posses activity selected from vasoactive action (e.g., vasoconstriction), hyperactivating platelets, pro-coagulant activities, and stimulating proliferation or production of thrombocytes or erythropoietin-dependent

(xv) The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

(xvi) The term “inflammatory conditions” as used herein refers to various diseases or traumas, whether mechanically or chemically induced, that have an inflammatory component. It includes conditions giving rise to inflammation in one or more organs or tissues including, but not limited to, the brain, spinal cord, connective tissue, heart, lung, kidney, urinary tract, pancreas, eyes and prostate. Non-limiting examples of such conditions include, but are not limited to, appendicitis, blepharitis, bronchitis, bursitis, cervicitis, cholangitis, cholecystitis, chorloamnionitis, conjunctivitis, cystitis, dacryoadenitis, dermatitis, endocarditis, endometritis, epicondylitis, epididymitis, fibrositis, gastritis, gingivitis, glossitis, hidradenitis suppurativa, iritis, laryngitis, mastitis, myocarditis, myositis, nephritis, omphalitis, oophoritis, orchids, osteitis, otitis, parotitis, pericarditis, peritonitis, pharyngitis, pleuritis, phlebitis, pneumonitis (pneumonia), prostatitis, pyelonephritis, rhinitis, salpingitis, sinusitis, stomatitis, synovitis, tonsillitis, uveitis, urethritis, vaginitis, vulvitis, asthma, systemic lupus erythematosus, myasthenia gravis, tendonitis, angiitis, chronic bronchitis, pancreatitis, osteomyelitis, arthritis (rheumatoid and psoriatic), glumeronephritis, optic neuritis, temporal arteritis, encephalitis, meningitis, traverse myelitis, dermatomyositis, polymyositis, necrodzing fasciitis, hepatitis, necrotizing entercolitis, pelvic inflammatory disease, inflammatory bowel disease (ulcerative colitis, Crohn's disease, ileitis, and enteritis), proctitis, vasculitis, vascular stenosis, restenosis, hypotension, Type-1 diabetes, Kawasaki disease, Decum's disease, chronic obstructive pulmonary disease, psoriasis, artherosclerosis, scieroderma, Sjogren's syndrome, mixed connective tissue disease, rosacea, gastric ulcers, duodenal ulcers, Alzheimer's disease, adult onset Still's disease, acute retinal pigment epitheliitis, Tietze's syndrome, Bechçet's disease, white dot syndrome (acute posterior multifocal placoid pigment epitheliopathy, serpiginous choroiditis, birdshot chorioretinopathy, multifocal choroiditis with panuveitis, diffuse subretinal fibrosis syndrome, punctuate inner choroidopathy, multiple evanescent white dot syndrome, and diffuse unilateral subacute neuroretinitis), granuloma annulate, irritable bowel syndrome, gastroenteritis, Grave's disease, multiple sclerosis, Dupuytren's contracture, graft rejection diseases (including allograft rejection and graft-v-host disease), e.g. skin graft rejection, solid organ transplant rejection, bone marrow transplant rejection, inflammatory dermatoses, viral cutaneous pathologies such as those derived from human papilloma virus, HIV, or RLV infection, bacterial, fungal and or other parasital cutaneous pathologies, cutaneous lupus erythematosus, and Hyper IgG4 disease. Further “Inflammatory condition” may refer to inflammation resulting from ischemic or non-ischemic conditions, including but not limited to, blunt trauma, contusions, allergies and allergic diseases, rheumatic disease (childhood arthritis, rheumatoid arthritis, Churg-Strauss syndrome, fibromyalgia giant cell (temporal) arteritis, gout, Henoch-Schoerdin purpura, hypersensitivity vasculitis, ankylosing spondylitis, capsulitis, rheumatic fever, rheumatic heart disease, systemic lupus erythematosus, polymyalgia rheumatica, osteoarthritis (hand, hip, knee, etc.) polyarteritis nodosa, Reiter's syndrome), sports related injuries (runner's knee, tennis elbow, frozen shoulders, chilles tendonitis, plantar fasciitis, bursitis, Osgood-Schlatter disease), repetitive stress injuries (cumulative trauma diseases, focal dystonia, carpal tunnel syndrome, intersection syndrome, reflex sympathetic dystrophy syndrome, stenosing tenosynovitis (De Quervain's syndrome, trigger finger/trigger thumb), thoracic outlet syndrome, tendonitis, tenosynovitis, radial tunnel syndrome, Raynaud's disease, ganglion, gamer's thumb, Wii-itis, etc.) infections including viral, fungal and bacterial. The “inflammatory condition” may be acute or chronic.

(xvii) An “isolated” or “purified” peptide is substantially free of cellular material or other contaminating proteins from the cell or tissue source from which the protein or peptide is derived, or substantially free of chemical precursors or other chemicals when chemically synthesized. The language “substantially free of cellular material” includes preparations of a peptide in which the peptide is separated from cellular components of the cells from which it is isolated or recombinantly produced. Thus, a peptide that is substantially free of cellular material includes preparations of peptides having less than about 30%, 20%, 10%, or 5% (by dry weight) of heterologous protein (also referred to herein as a “contaminating protein”). When the peptide is recombinantly produced, it is also preferably substantially free of culture medium, i.e., culture medium represents less than about 20%, 10%, or 5% of the volume of the protein preparation. When the peptide is produced by chemical synthesis, it is preferably substantially free of chemical precursors or other chemicals, i.e., it is separated from chemical precursors or other chemicals which are involved in the synthesis of the protein. Accordingly such preparations of the peptide have less than about 30%, 20%, 10%, 5% (by dry weight) of chemical precursors or compounds other than the peptide of interest. In a preferred embodiment, peptides of the invention are isolated or purified.

(xviii) An “isolated” nucleic acid molecule is one which is separated from other nucleic acid molecules which are present in the natural source of the nucleic acid molecule. Moreover, an “isolated” nucleic acid molecule, such as a cDNA molecule, can he substantially free of other cellular material, or culture medium when produced by recombinant techniques, or substantially free of chemical precursors or other chemicals when chemically synthesized. In specific embodiment, a nucleic acid molecule(s) encoding a peptide of the invention is isolated or purified.

(xix) As used herein, the term “management” includes the provision of one or more beneficial side effects that a patient derives from a peptide which, in one embodiment, does not reverse the damage, effects or symptoms of a a disease or disorder associated with tissue damage. In certain embodiments, a patient is administered a peptide to “manage” the symptoms of a disease or disorder associated with tissue damage so as to prevent the progression or worsening of the symptoms.

(xx) The terms “modulate,” “modulations” and the like refer to the ability of a compound to increase or decrease the function and/or expression of mediators of the body's response to a disease or disorder associated with tissue damage, including transcription of regulatory activity and/or protein binding. Modulation, as described herein, includes the inhibition, antagonism, partial antagonism, activation, agonism or partial agonism of a function or characteristic associated with the mediator, either directly or indirectly, and/or the upregulation or downregulation of the expression of the mediator. In a preferred embodiment, the modulation is direct, and more preferably the modulation occurs through an inhibitor or antagonist of the mediator, a compound that binds to, partially or totally blocks stimulation, decreases, prevents, inhibits, delays activation, inactivates, desensitizes, or downregulates signal transduction. The ability of a particular peptide useful in the method of the current invention to inhibit the function of a mediator can be demonstrated in a biochemical assay, e.g. binding assay, cell based assay, e.g. transient transfection assay, or in vivo assay, e.g. animal model of neuronal injury, cancer, inflammation, or chemical or radiation injury such as a rat or murine model.

(xxi) As used herein in reference to a structure within a peptide, the term “motif” refers either to a set of consecutive amino acids within the amino acid sequence of the peptide chain and/or to a set of linearly or spatially adjacent amino acids within the secondary and/or tertiary structure of said peptide. Because the motif may be formed all or in part as a result of protein ridding, amino acids that arc adjacent in the described motif may be separated by 0, 1 or more, 5 or more, 10 or more, 15 or more or 20 or more amino acids within the linear amino acid sequence of the peptide.

(xxii) As used herein, the terms “peptide,” “polypeptide” and “protein” are used interchangeably and in their broadest sense to refer to constrained (that is, having some element of structure as, for example, the presence of amino acids which initiate a β turn or β pleated sheet, or for example, cyclized by the presence of disulfide bonded Cys residues) or unconstrained (e.g., linear) amino acid sequences. In certain embodiments, the peptide of the invention consists of less than 30 amino acids. However, upon reading the instant disclosure, the skilled artisan will recognize that it is not the length of a particular peptide but its ability to bind a tissue protective receptor complex and/or compete with the binding of a peptide described herein that distinguishes the peptides useful in the method of the current invention. The terms “peptide,” “polypeptide,” and “protein” also refer to compounds containing amino acid equivalents or other non-amino acid groups, while still retaining the desired functional activity of a peptide or protein. Peptide equivalents can differ from conventional peptides by the replacement of one or more amino acids with related organic acids (such as PABA), amino acid. equivalents or the like or the substitution or modification of side chains or functional groups.

(xxiii) The term “preventing the damages, effects or symptoms of a disease or disorder associated with tissue damage” means delaying the onset, hindering, the progress, hindering the appearance, protection against, inhibiting or eliminating the emergence, or reducing the incidence, of such damages, effects or symptoms. Use of the term “prevention” is not meant to imply that all patients in a patient population administered a preventative therapy will never be affected by or develop symptoms in response to the disease or disorder associated with tissue damage targeted for prevention, but rather that the patient population will exhibit a reduction in the damage, effects, or symptoms of the disease or disorder. For example, many flu vaccines are not 100% effective at preventing flu in those administered the vaccine. One skilled in the art can readily identify patients and situations for whom preventative therapy would be beneficial, such as, but not limited to, individuals about to engage in activities that may expose them to various toxic agents or traumas (e.g., soldiers engaging in military operations, chemical or food processing workers, emergency personnel or first responders, etc.), or individuals that may be subjected to exposure to a toxic agent (e.g., individuals living in the vicinity of chemical, nuclear, or manufacturing facilities, or individuals under threat of military or terrorist attack).

(xxiv) As used herein, a “prophylactically effective amount” refers to that amount of a peptide sufficient to result in the prevention of the damage, effects or symptoms resulting from a disease or disorder associated with tissue damage. A prophylactically effective amount can refer to the amount of a peptide sufficient to prevent the damage, effects or symptoms resulting from a disease or disorder associated with tissue damage. Further, a prophylactically effective amount with respect to another prophylactic agent means that amount of that prophylactic agent in combination with a peptide that provides a prophylactic benefit in the prevention of damage, effects or symptoms resulting from a disease or disorder associated with tissue damage. Used in connection with an amount. of a peptide, the term “prophylactically effective amount” can encompass an amount that improves overall prophylaxis or enhances the prophylactic efficacy of or provides a synergistic affect with another prophylactic agent.

(xxv) The term “neoplasm” refers to abnormal growths that lack the malignant properties of cancerous tumors, and are generally mild and non-progressive tumors. Neoplasms, include but are not limited to moles, uterine fibroids, thyroid adenomas, adrenocortical adenomas, pituitary adenomas, and teratomas.

(xxvi) The term “radiation agent” as used herein means any radioactive material that may kill or injure a subject, and may be used to cause disruption upon a city or nation. Exposure to a radiation agent may occur through deployment of a weapon (nuclear bomb (fission, fusion, neutron, boosted fission, or salted bombs), shells containing depleted uranium), terrorist device (“dirty bomb”), or fallout resulting from the detonation of a nuclear weapon or failure of a nuclear plant. Radioactive agents may include, but are not limited to, ¹³⁷Cs, ⁶⁰Co, ²⁴¹Am, ²¹²Cf, ¹⁹²Ir, ²³⁸Py, ⁹⁰Sr, ²²⁶Ra, ⁹¹Sr, ⁹²Sr, ⁹⁵Zr, ⁹⁹Mo, ¹⁰⁶Ru, ¹³¹Sb, ¹³²Te, ¹³⁹Te, ¹⁴⁰Ba, ¹⁴¹La, ¹⁴⁴Ce, ²³³U, ²³⁵U, ²³⁸U, ²²⁸P, ²²⁹P, ²³⁰P, ²³¹P, ²³²P, ²³³P, ²³⁴P, ²³⁵P, ²³⁶P, ²³⁷P, ²³⁸P, ²³⁹P, ²⁴⁰P, ²⁴¹P, ²⁴²P, ²⁴³P, ²⁴⁴P, ²⁴⁵P, ²⁴⁶P, ²⁴⁷P, and ¹³¹I. Exposure to the radioactive agents can result in carcinogenesis, sterilization, cataract formation, radiodermatitis, beta burns, gamma burns, loss of cells (in particular bone marrow, digestive tract cells), damage to the hematopoietic, gastrointestinal, central nervous, cardiovascular, skin, and/or reproductive systems, acute radiation syndrome, chronic radiation syndrome, and cutaneous radiation syndrome. Acute radiation syndrome generally results from large doses of radiation to a subject's body occurring in a short period of time. The syndrome has a predictable course starting with a feeling of nausea, vomiting, general illness and fatigue, immune system depression, loss of hair, uncontrollable bleeding (mouth, under the skin, kidneys), massive diarrhea, delirium, coma and death. Cutaneous radiation syndrome is a subset of acute radiation syndrome and refers to radiations effects on the skin, which include, but are not limited to, inflammation, erythema, dry or moist desquamation, hair loss, blistering, reddening, ulceration, damage to sebaceous and sweat glands, atrophy, fibrosis, decreased or increased skin pigmentation, and necrosis.

(xxvii) As used herein, the terms “subject,” “patient” and “victim” are used interchangeably. As used herein, the terms “subject” and “subjects” refer to an animal, preferably a mammal including a non-primate (e.g., a cow, pig, horse, cat, dog, rat, and mouse and a primate (e.g., a monkey, ape, or a human), and more preferably a human.

(xxviii) As used herein, the term “syndromes associated with neoplasms or cancers” refers to syndromes resulting from the direct action of the tumors through “mass effect” (compression of vital organs due to tumor) or “functional tumors” (overproduction of hormones by organ afflicted with tumor). Such syndromes include, but are not limited to, Beckwith-Wiedmann syndrome, SBLA syndrome, Li-Fraumeni syndrome, Familial Adenomatous Polyposis syndrome (Gardner syndrome), Hereditary Nonpolyposis Colorectal Cancer, Turcot syndrome, Cowden syndrome, Carney Triad syndrome, Multiple Endocrine Neoplasia syndromes (Wermer (MEN-1), Sipple (MEN-2a, MEN-2b), Von Hipple-Lindau syndrome, Cushing's syndrome, Addison's syndrome, Verner Morrison syndrome, Zollinger-Ellison syndrome, WDHA syndrome, Pancreatic Cholera, Isaac's syndrome, Rippling muscle syndrome, Stiffman syndrome, Paraneoplastic Ataxia, Yo syndrome, Tr syndrome, Hu syndrome, CV-2 syndrome, CRMP-5 syndromes, Opsoclonus/Myoclonus, Ma syndromes, Morvan's fibrillary chorea, Bannayan-Riley-Runalcaba syndrome, Peutz-Jegher syndrome, Muir-Torre syndrome, Hirschsprung disease, Lynch syndrome, Lambert-Eaton Myastenic syndrome, Myasthenia Gravis, Neuromyotonia, Paraneoplastic Cembellat Degeneration, Paraneoplastic Limbic Encephalitis, Sweets syndrome, Birt-Hogg-Dube syndrome, Naevoid Basal Cell Carcinoma syndrome, Generalized Basaloid Follicular syndrome, Hamartoma syndrome, Bazex syndrome, Brooke Spiegler syndrome, Familial Cylindromatosis, Multiple Familial Trichoepitheliomas, Androgen Deprivation syndrome, Therapy Related Myelodysplastic syndrome, Somnolence syndrome, Gulf War syndrome, and Somatostatinoma syndrome.

(xxvix) As used herein, the term “tissue protective activity” or “tissue protection” refers to the effect of inhibiting or delaying damage or death of a cell, tissue, or organ. Unless otherwise noted, the “delay” in damage or death of a cell, tissue or organ is evaluated relative to a control condition in the absence of a peptide of the invention. Tissue protective activity is specific to tissue, cells, and/or organs expressing a tissue protective receptor complex (i.e., a responsive tissue cell, and/or organ, respectively), such as, but not limited to, the tissues of the central nervous system. In specific embodiments, the responsive cells are not erythrocyte progenitor cells.

(xxx) The term “tissue protective receptor complex” as used herein means a complex comprising at least one erythropoietin receptor subunit and at least one beta common receptor subunit. The tissue protective receptor complex may contain multiple erythropoietin receptor subunits and/or beta common receptor subunits, as well as other types of receptors or proteins. See WO 20041096148, which is hereby incorporated by reference herein in its entirety.

(xxxi) The term “toxic agent” as used herein refers to the biological, chemical and radiation agents mentioned above.

(xxxii) To determine the percent identity of two amino acid sequences, the sequences are aligned for optimal comparison purposes. The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical overlapping positions×100/total number of positions). In one embodiment, the two sequences are the same length, in an alternate embodiment, the sequences are of different length and, accordingly, the percent identity refers to a comparison of the shorter sequence to a portion of the longer sequence, wherein said portion is the same length as said shorter sequence.

(xxxiii) As used herein, the term “treatment” includes the elimination, reduction, management or control of damage, effects or symptoms resulting from a disease or disorder associated with tissue damage or the damage, effects, or symptoms thereof.

### 5. BRIEF DESCRIPTION OF THE FIGURES

FIG. 1 shows the effect of a peptide of the invention in a sciatic nerve injury model in the rat.

FIG. 2 shows the effect of a peptide of the invention on the induction of edema by intradermal histamine injection in a rat model.

FIG. 3A-D show the effect of a peptide of the invention on (A) weight loss, (B) epididymal fat, (C) lean mass and (D) physical activity in a cachexia model.

### 6. DETAILED DESCRIPTION OF THE INVENTION

6.1 Isolated Polypeptides

The current invention provides a method of modulating the effects of the body's response to a disease or disorder associated with tissue damage. Further, the current invention provides a method of preventing, treating, ameliorating, or managing damage, effects or symptoms in a patient afflicted with a disease or disorder associated with tissue damage by administering a peptide that is derived from erythropoietin or another Type-1 cytokine. Preferably, the peptide used in the current method is tissue protective, neuroprotective, neuritogenic, or anti-apoptotic.

Several peptides derived from Type-1 cytokines, such as EPO, have been disclosed in the art, such as: (a) U.S Pat. No. 8,071,554, issued Dec. 6, 2011, U.S. Publication Nos: 2012-0142595, published Jun. 7, 2012, 2012-0264682, published Oct. 18, 2012, all to Cerami et al.; (b) U.S. Publication. No. 2011-0263504, published Oct. 27, 2011, to Cerami et al.; (c) Bock et al. in PCT Application No. DK2006/000246 published as WO 2006/119767 and WO 2007/071248 (“Bock”); (d) O'Brien et al. in U.S. Pat. Nos. 5,571,787, 5,700,909, 5,696,080, 5,714,459, 6,590,074, 6,559,124, 6,271,196, 6,268,347, and 6,849,602 (“O'Brien”); (e) Smith-Swintowsky et al. in U.S. Pat. No. 7,259,146 and U.S. Publication No. 20030130197(“Smith-Swintowsky”), and (f) Yuan et al. in PCT/IB2006/003581 published as WO/2007/052154 (“Yuan”) each of these inventions is hereby incorporated in their entirety.

As stated above, peptides useful for the modulation of the body's response to a disease or disorder associated with tissue damage and/or useful in the prevention, treatment, amelioration and management of damage, effects or symptoms in a subject afflicted with a disease or disorder associated with tissue damage have a motif based, in one embodiment, on the amino acid sequence RYLLEAKEAENITTG (SEQ ID NO:1) or a peptide comprising the amino acid sequence RYLLEAKEAENITTG (SEQ ID NO:1).

The isolated peptide of the invention having the amino acid sequence RYLLEAKEAENITTG (SEQ ID NO:1) can also be extended at one or both termini or internally with additional amino acid residues that do not substantially interfere with, and in some embodiments even enhance, the structural and/or functional properties of the peptides or peptide analogs. Indeed, extended core peptides and peptide analogs containing as many as 16, 17, 18, 19, 20, 21, 22, 21, 24, 25, 26, 27, 28, or 29 amino acid residues and are considered to be within the scope of the present invention. However, in one aspect of the invention, the extended core peptides or peptide analogs of the invention do not have the amino acid sequence of RYLLEAKEAENITTGC (SEQ ID NO: 2), or the extended core peptides or peptide analogs of the invention do not have an amino acid sequence of EPO outside of SEQ ID NO:1.

6.3 Fusion Peptides

The present invention further contemplates that two or more of the above noted peptide may be linked to a related or unrelated protein such as erythropoietin, albumin, etc. Such fusion peptides may be generated in order to achieve synergistic benefits, increase the circulating half-life of the peptide, or increase the ability of the peptide to penetrate endothelial barriers, such as the blood-brain barrier, blood-retina barrier, etc., or vice versa, i.e. to act as a transport mechanism similar to that disclosed in PCT/US01/49479 published as WO 2002/053580 hereby incorporated in its entirety by reference.

6.5 Manufacture of Peptides

Peptides useful in the method of the current invention may be made using recombinant or synthetic techniques well known in the art. In particular, solid phase protein synthesis is well suited to the relatively short length of the peptides and may provide greater yields with more consistent results. Additionally, the solid phase protein synthesis may provide additional flexibility regarding the manufacture of the peptides. For example, desired chemical modifications may be incorporated into the peptide at the synthesis stage: homocitrulline could be used in the synthesis of the peptide as opposed to lysine, thereby obviating the need to carbamylate the peptide following synthesis or amino acids with protected functional groups may be left on the peptide during synthesis.

Synthesis

The isolated peptides and peptide analogs useful in the method of the current invention may be prepared using conventional step-wise solution or solid phase synthesis (see, e.g., Merrifield, R. B., 1963, J. Am. Chem. Soc. 85:2149-2154; Chemical Approaches to the Synthesis of Peptides and Proteins, Williams et al.. Eds., 1997, CRC Press, Boca Raton Fla., and references cited therein; Solid Phase Peptide Synthesis: A Practical Approach, Atherton & Sheppard, Eds., 1989, IRL Press, Oxford, England, and references cited therein).

Alternatively, the peptides and peptide analogs useful in the current invention may be prepared by way of segment condensation, as described, for example, in Liu et al., 1996, Tetrahedron Lett, 37(7):933-936; Baca, et al., 1995, J. Chem Soc. 117:1881-1887; Tam et al., 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, Proc. Natl. Acad. Sci. USA 91:0584-6588; Yamashiro and Li, 1988, Int Peptide Protein Res. 31:322-334). This is particularly the case with Gly (G) containing peptides. Other methods useful for synthesizing the peptides and peptide analogs of the invention are described in Nakagawa et al., 1985, J. Am. Chem. Soc. 107:7087-7092.

Recombinant Techniques

A variety of host-expression vector systems may be utilized to produce the peptides and peptide analogues. Such host-expression systems represent vehicles by which the peptide of interest may be produced and subsequently purified, but also represent cells that may, when transformed or transfected with the appropriate nucleotide coding sequences, exhibit the modified erythropoietin gene product in situ. These include hut are not limited to, bacteria, insect, plant, mammalian, including human host systems, such as, but not limited to, insect cell systems infected with recombinant virus expression vectors (e.g., baculovirus) containing the peptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing erythropoietin-related molecule coding sequences; or mammalian cell systems, including human cell systems, e.g., HT1080, COS, CHO, BM, 293, 3T3, PERC6 harboring recombinant expression constructs containing promoters derived from the genome of mammalian cells, e.g., metallothionein promoter, or from mammalian viruses, e.g., the adenovirus late promoter; the vaccinia virus 7.5K promoter.

In addition, a host cell strain may be chosen that modulates the expression of the inserted sequences, or modifies and processes the gene product in the specific fashion desired. Such modifications and processing of protein products may be important for the function of the protein. As known to those of ordinary skill in the art, different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and W138.

For long-term, high-yield production of recombinant peptides, stable expression is preferred. For example, cell lines that stably express the recombinant tissue protective cytokine-related molecule gene product may be engineered. Rather than using expression vectors that contain viral origins of replication, host cells can be transformed with DNA controlled by appropriate expression control elements, e,g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously he used to engineer cell lines that express the tissue-protective product. Such engineered cell lines may he particularly useful in screening and evaluation of compounds that affect the endogenous activity of the EPO-related molecule gene product.

Further Modifications

Peptides with additional modifications can also be used in the method of the present invention for preventing, treating, ameliorating or managing a disease or disorder associated with tissue damage or damage, effects or symptoms resulting therefrom. For example, the peptides of the above-noted structural motifs may be synthesized with one or more (D)-amino acids. The choice of including an (L)- or (D)-amino acid into a peptide of the present invention depends, in part, upon the desired characteristics of the peptide. For example, the incoporation of one or more (D)-amino acids can confer increasing stability on the peptide in vitro or in vivo. The incorporation of one or more (D)-amino acids can also increase or decrease the binding activity of the peptide as determined, for example, using the bioassays described herein, or other methods well known in the art.

Replacement of all or part of a sequence of (L)-amino acids by the respective sequence of entatiomeric (D)-amino acids renders an optically isomeric structure in the respective part of the peptide chain inversion of the sequence of all or part of a sequence of (L)-amino acids renders retro-analogues of the peptide. Combination of the enantiomeric (L to D, or D to L) replacement and inversion of the sequence renders retro-inverso-analogues of the peptide. It is known to those skilled in the art that enantiomeric peptides, their retro-analogues, and their retro-inverso-analogues maintain significant topological relationship to the parent peptide, and especially high degree of resemblance is often obtained for the parent and its retro-inverse-analogues. This relationship and resemblance can he reflected in biochemical properties of the peptides, especially high degrees of binding of the respective peptides and analogs to a receptor protein. The synthesis of the properties of retro-inverso anologues of peptides have been discussed for example in Methods of Organic Chemistry (Houben-Weyl), Synthesis of Peptides and Peptidomimetics—Workbench Edition Volume E22c (Editor-in-chief Goodman M.) 2004 (George Thieme Verlag Stuttgart, New York), and in references cited therein, all of which are hereby incorporated by reference herein in their entireties.

Amino acid “modification” refers to the alteration of a naturally occurring amino acid to produce a non-naturally occurring amino acid. Derivatives of the peptides of the present invention with non-naturally occurring amino acids can be created by chemical synthesis or by site specific incorporation of unnatural amino acids into peptides during biosynthesis, as described in Christopher J. Noren, Spencer J. Anthony-Cahill, Michael C. Griffith, Peter G. Schultz, 1989 Science, 244:182-188, hereby incorporated by reference herein in its entirety.

Peptide mimetics that are structurally similar to therapeutically useful peptides may he used to produce an equivalent therapeutic or prophylactic effect. Generally, peptidomirnetics are structurally similar to a paradigm polypeptide (i.e., a polypeptide that has a biochemical property or pharmacological activity), but have one or more peptide linkages optionally replaced by a linkage selected from the group consisting of: —CH₂—NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p 267 (1983); Spatula, A. F., Vega Data (March 1983), Vol. 1. Issue 3, “Peptide Backbone Modifications” (general review); Merely, J. S., Trends Pharma Sci (1980) pp. 463-468 (general review); Hudson, D. et al., (1979) Int J Pept Prot Re 14; 177-185 (—CH₂—NH—, —CH₂—CH₂—); Spatula, A. F. et al., (1986) Life Sci 38:1243-1249 (—CH₂—S—); Harm, M. M., (1982) J Chem Soc Perkin Trans 1 307-314 cis and trans); Almquist, R. G. et al., (1980) J Med Chem 23: 1392 (—COCH₂—); Jennings-White, C et al,, (1982) Tetrahedron Lett 23:2533 (—COCH₂—); Szeike, M et al., European Appln. EP 45665 (1982) CA: 97: 39405 (1982) (—CH(OH)CH₂—); Holladay, M. W. et al., (1983) Tetrahedron Lett 24:4401-4404 (—C(OH)CH₂—); and Hruby, V J., (1982) Life Sci 31:189-199 (—CH₂ S—); each of which is incorporated herein by reference.

In another embodiment, a particularly preferred non-peptide linkage is —CH₂NH—. Such peptide mimetics may have significant advantages over peptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

A variety of designs for peptide mimetics are possible. For example, cyclic peptides, in which the necessary conformation is stabilized by non-peptides, are specifically contemplated, U.S. Pat. No. 5,192,746 to Lobl, et al., U.S. Pat. No. 5,576,423 to Aversa, et al., U.S. Pat. No. 5,051.448 to Shashoua, and U.S. Pat. No. 5,559,103 to Gaeta, et al., all hereby incorporated by reference, describe multiple methods for creating such compounds. Synthesis of nonpeptide compounds that mimic peptide sequences is also known in the art. Eldred et al., J. Med. Chem. 37:3882 (1994), hereby incorporated by reference herein in its entirety) describe non-peptide antagonists that mimic the peptide sequence. Likewise, Ku et al., J. Med. Chem 38:9 (1995) (hereby incorporated by reference herein in its entirety) further elucidates the synthesis of a series of such compounds.

Further modifications following synthesis may be implemented. For example, the peptides may be farther chemically modified, i.e. carbamylated, acetylated, succinylated, guanidated, nitrated, trinitrophenylated, amidinated, etc., in accordance with U.S. patent application Ser. No. 10/188,905, which published as 20030072737-A1 on Apr. 17, 2003 and discloses chemically modified EPO, and in accordance with U.S. patent application Ser. No. 10/612,665, filed Jul. 1, 2003, and U.S. patent application Ser. No. 09/753,132, tiled Dec. 29, 2000, which are incorporated by reference herein in their entirety.

Additionally, the peptides may consist of recombinant peptides—muteins. The disclosed mutations may include substitutions, deletions, including internal deletions, additions, including additions yielding fusion proteins, or conservative substitutions of amino acid residues within and/or adjacent to the amino acid sequence, but that result in a “silent” change, and non-conservative amino acid changes and larger insertions and deletions, as previously disclosed in PCT/US03120964 entitled Recombinant Tissue Protective Cytokines and Encoding Nucleic Acids Thereof for Protection, Restoration, and Enhancement of Responsive Cells, Tissues, and Organs (which is incorporated by reference herein in its entirety)

Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids can be divided into four families: (1) acidic=Asp (D), Glu (G) (2) basic=Lys (K), Arg (R), His (H); (3) nonpolar (hydrophobic)=Cys (C), Ala (A), Val (V), Leu (L), Ile (1), Pro (P), Phe (F), Met (M), Trp (W), Gly (G), Tyr (Y); and (4) uncharged polar Asn (N), Gin (Q), Ser (S), Thr (T). Non-polar may be subdivided into: strongly hydrophobic=Ala (A), Val (V), Leu (L), Ile (I), Met (M), Phe (F); and moderately hydrophobic=Gly (C), Pro (P), Cys (C), Tyr (Y), Trp (W). In alternative fashion, the amino acid repertoire can be grouped as (1) acidic=Asp (D), Gln (C); (2) basic=Lys (K), Are (R), His (H), (3) aliphatic=Gly (G), Ala (A), Val (V), Leu (L), Ile (I), Ser (S), Thr (T), with Ser (S) and Thr (T) optionally be grouped separately as aliphatic-hydroxyl; (4) aromatic=Phe (F), Tyr (Y), Trp (W); (5) amide=Asn (N), Gln (Q); and (6) sulfur-containing=Cys (C) and Met (M). (See, for example, Biochemistry, 4th ed., Ed. by L. Stryer, WH Freeman and Co., 1995, which is incorporated by reference herein in its entirety).

Alternatively, mutations can be introduced randomly along all or part of the coding sequence of a peptide, such as by saturation mutagenesis, and the resultant mutants can be screened for biological activity to identify mutants that retain activity. Following mutagenesis, the encoded peptide can be expressed recombinantly and the activity of the recombinant peptide can be determined.

In another embodiment, the peptide may be further modified through the additions of polymers (such as polyethylene glycol), sugars, or additional proteins (such as a fusion construct) in an effort to extend the half-life of the peptide or enhance the peptide's tissue protective effects. Examples of such modifications are disclosed within WO/04022577 A3 and WO105025606 A1, which are incorporated herein by reference. For example, a polyethylene glycol polymer can be attached to Peptide IC to result in a Peptide IW (see, SEQ ID NO. 298; PEG-QEOLERALNSS (herein SEQ ID NO: 3) in WO105025606 A1).

Depending, on the conjugation chemistry selected and the number of reactive sites already present or created on the peptide, one, two, or a selected number of polymers can be appended in a reproducible manner. The principal mode of attachment of a PEG, and its derivatives, to peptides is a non-specific bonding through a peptide amino acid residue (see e.g., U.S. Pat. No. 4,088,538, U.S. Pat. No. 4,496,689, U.S. Pat. No. 4,414,147, U.S. Pat. No. 4,055,635, and PCT WO 87/00056). Another mode of attaching PEG to peptides is through the non-specific oxidation of glycosyl residues on a glycopeptide (see e.g., WO 94/05332). In these non-specific methods, PEG is added in a random, non-specific manner to reactive residues on a peptide backbone.

### 7. ASSAYS FOR TESTINC PEPTIDES

Various assays can be used to determine the utility of the above noted peptides for use in the therapeutic methods of the current invention. The peptides tissue protective activity would be confirmed using various assays known in the art and disclosed within U.S. patent application Ser. Nos. 10/554,517, 10/612,665 and 11/997,898. Further, the peptides lack of erythropoietic activity or reduced erythropoietic activity will be confirmed using various in vitro assays such as EPO dependent cell lines (UT-7), mouse spleen bioassay (Krystal. G. (1983) (a simple microassay for erythropoietin based on ³H-thymidine incorporation into spleen cells for phenylhydrazine treated mice. Exp. Hematol. 11, 649-660)), or clonal assays (Spivak, J. L., Seiber, F. (1983). Erythropoietin. Horm. Norm. Abnorm. Hum. Tissues 3, 63-96), and in vivo assays, such as the ex hypoxic polycythemic mouse assay (Cotes P M, Bangham D R, Bio-assay of erythropoietin in mice made polycythaemic by exposure to air at a reduced pressure, Nature. 1961 Sep. 9; 191:1065-7). Additionally, one of ordinary skill in the art will recognize that the peptide's ability to prevent, mitigate or treat a disease or disorder associated with tissue damage or damage, effects or symptoms resulting, therefrom may be confirmed through various assays both in vitro and in vivo, although in certain embodiments in vivo assays may be preferred.

7.1 Tissue Protective Assays and Models

The peptides utilized in the current method exhibit tissue protective properties, i.e. anti-apoptoitc, neuritogenic, neuroprotective, anti-cachectic, anti-inflammatory etc. Peptides in accordance with the present invention may be tested for tissue protective activity, e.g., protecting cells, tissues or organs. Protective activities may be further tested using in vitro and in vivo assays. In vitro tests that are indicative of tissue protective activity include, for example, cell proliferation assays, cell differentiation assays, or detecting the presence of proteins or nucleic acids upregulated by tissue protective receptor complex, e.g. tissue protective cytokine receptor complex, activity, e.g., nucleolin, neuroglobin, cytoglobin, or frataxin. Neuroglobin, for example, may be involved in facilitating the transport or the short-terms storage of oxygen. Therefore, oxygen transport or storage assays may be used as an assay to identify or screen for compounds which modulate tissue protective activity.

Neuroglobin is expressed in cells and tissues of the central nervous system in response to hypoxia or ischemia and may provide protection from injury (Sun et al. 2001, PNAS 98:15306-15311; Schmid et al., 2003, J. Biol. Chem, 276:1932-1935, each of which is incorporated by reference herein in its entirety). Cytoglobin may play a similar role in protection, but is expressed in a variety of tissues at varying levels (Pesce et al., 2002, EMBO 3:1146-1151, which is incorporated by reference herein in its entirety). In one embodiment of the invention, the levels of an upregulated protein in a cell may be measured before and after contacting the peptide to a cell. In certain embodiments, the presence of an upregulated protein associated with tissue protective activity in a cell, may be used to confirm the tissue protective activities of a peptide.

Nucteolin may protect cells from damage. It plays numerous roles in cells including modulation of transcription processes, sequence specific RNA-binding protein, cytokinesis, nucleogensis, signal transduction, apoptosis induced by T-cells, chromatin remodelling, or replication. It can also function as a cell surface receptor DNA/RNA helicase, DNA-dependent ATPase, protein shuttle, transcription factor component, or transcriptional repressor (Srivastava and Pollard, 1999, FASEB 13:1911-192.2; and Ginisty el al., 1999, J. Cell Sci., 112:761-772, each of which is incorporated by reference herein in its entirety).

Frataxin is a protein involved with mitochondrial iron metabolism and has previously been shown to be strongly up-regulated by EPO both in vivo and in vitro (Sturm et al. (2005) Eur J Clin Invest 35: 711, which is incorporated by reference herein in its entirety)

Expression of an upregulated protein may be detected by detecting mRNA levels corresponding to the protein in a cell. The mRNA can be hybridized to a probe that specifically binds a nucleic acid encoding the upregulated protein. Hybridization may consist of, for example, Northern blot, Southern blot, array hybridization, affinity chromatography, or in situ hybridization.

Tissue protective activity of the peptide of the invention can also be detected using in vitro neuroprotection assays. For example, primary neuronal cultures may be prepared from new born rat hippocampi by trypsinization, and cultured as by any method known in the art and/or described herein e.g. in MEM-II growth medium (Invitrogen), 20 mM D-glucose, 2 mM L-glutamine, 10% Nu-serum (bovine; Becton. Dickinson, Franklin Lakes, N.J.), 2% B27 supplement (Invitrogen), 26.2 mM NaHCO3, 100 U/ml penicillin, and 1 mg/ml streptavidin (see, e.g., Leist et al., 2004, Science 305:239-242, hereby incorporated by reference in its entirety). One day after seeding, 1 μM cytosinearabino-furanoside is added. Thirteen day old cultures are then preincubated with increasing doses of the peptide of interest (3-3000 pM) for 24 h. On day 14, the medium is removed and the cultures challenged with 300 μM NMDA in PBS at room temperature (RT). After 5 min, pre-conditioned medium is rotumed to the cultures which are then returned to the incubator for 24 h, The cells are fixed in paraformaldehyde, stained by Hoechst 33342 (Molecular Probes, Eugene, Oreg.) and condensed apoptotic nuclei may be counted, NGF (50 ng/ml) and MK801 (1 μM) are included as positive controls.

Animal model systems can be used to demonstrate the tissue protective activity of a compound or to demonstrate the safety and efficacy of the compounds identified by the screening methods of the invention described above. The compounds identified in the assays can then be tested for biological activity using animal models for a type of tissue damage, disease, condition, or syndrome of interest. These include animals engineered to contain the tissue protective receptor complex coupled to a functional readout system, such as a transgenic mouse.

Animal models that can be used to test the efficacy of the cell or tissue protective activity of an identified compound are known in the art and include, for example, protection against the onset of acute experimental allergic encephalomyelitis in Lewis rats, restoration or protection from diminished cognitive function in mice after receiving brain trauma, cerebral ischemia (“stroke”) or seizures stimulated by excitotoxins (Brines et al., 2000, PNAS, 97:10295-10672, which is incorporated by reference herein in its entirety), protection from induced retinal ischemia (Rosenbaum et al., 1997, Vis. Res. 37:3443-51 which is incorporated by reference herein in its entirety), protection from injury to the sciatic, nerve, and protection from ischemia-reperfusion injury to the heart (in vitro cardiomyocyte studies and in vivo ischemia-reperfusion injury, see, e.g., Calvillo et al., 2003, PNA 100:4802-4806 and Fiordaliso et al., 2005, PNAS 102:2046-2051, each of which is hereby incorporated by reference in its entirety). Such assays are described in further detail in Grasso et al. (2004) Med Sci Monit 10: BR2-3, PCT publication no. WO02/053580, or PCT application PCT/US2006/031061 each of which is incorporated by reference herein in its entirety. The in vivo methods described therein are directed towards administration of EPO, however, tissue protective proteins administered in place of EPO have been identified to also exhibit similar biologic activity, e.g., Leist et al, (2004) Science 305: 239-242, which is incorporated by reference herein in its entirety. Peptides may be substituted for testing as well. Other assays for determining tissue protective activity of a peptide are well known to those of skill in the art.

Alternatively, cell binding assays can be for evaluation of the peptides of the invention. For example, the peptide of interest can be bound to a biological marker such as a fluorescent or radiolabled marker for ease of detection and tested for binding to transfected BaF3 cells expressing EPOR and/or β_{c} receptor. In a 96 well plate, eight 1:2 serial dilutions of the peptide of interest in growth medium (RPMI 1640, 10% fetal bovine serum, 1 mM sodium pyruvate, 2 mM L-glutamine) are plated, such that the final volume in each well is about 100 μl. The BaF3 parental line and BaF3 cells transfected with EPOR and/or β_{c} receptor can be washed three times in growth media (see above), pellets resuspended in growth medium, and cells counted and diluted in growth media to 5,000 cells/100 μl. 100 μl of diluted cells are then added to each peptide dilution. The assay plate is then incubated in a 37° C. incubator for three to four days. The plate/cells are then washed and the plate is read on a fluorescent plate reader or by other suitable method to detect the level of biomarker associated with the biological activity of the peptide of interest.

Similarly, a competitive assay can be utilized to determine if a peptide is tissue protective. In the competitive assay, a compound known to be tissue protective including, but not limited to, tissue protective cytokines such as those disclosed in U.S. patent application Ser. Nos. 10/188,905 and 10/185,841 (each of which is incorporated by reference herein in its entirety), can be attached to a suitable bio marker.

In a 96 well plate eight 1:2 serial dilutions of a known tissue protective compound/biomarker in suitable growth medium, and the same dilution series of the known tissue protective compound/biomarker and an excess of the peptide of interest are plated. The Final volume of each dilution should be about 100 μl. Once again, the BaF3 cells are seeded into the plates as disclosed supra and allowed to incubate. After an appropriate amount of time, the cells are washed and the plate is read on a fluorescent plate reader or by any other suitable method known in the art to detect the biomarker. If the readout of the plates and/or wells containing the known tissue protective compound/biomarker and peptide of interest is less than the readout of the plates containing only the known tissue protective compound/biomarker then the peptide of interest is tissue protective.

Many protein factors discovered to date, including all known cytokines, have exhibited activity in one or more factor-dependent cell proliferation assays, and hence these assays serve as a convenient confirmation of cytokine activity. The activity of a peptide can be evidenced by any one of a number of routine factor dependent cell proliferation assays for cell lines including, without limitation, 32D, DA2, DA1G, T10, B9, B9/11, BaF3, MC9/G, M+(preB M+). 2E8, RB5, DA1, 123, T1165, HT2, CTL1,2, TF-1, Mo7e and CMK. These cells are cultured in the presence or absence of a peptide, and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65:55-63, which is incorporated by reference herein in its entirety).

If a peptide exhibits a tissue protective activity one of ordinary skill in the art would recognize that it would be beneficial to verify the result using one of the neuroprotective and tissue protective assays known to those skilled in the art, such as, but not limited to, P-19 and PC-12 cell assays. Additionally, various in vivo models such as animal models related to spinal cord injury, ischemic stroke, peripheral nerve damage, wounds, or damage to the heart, eyes, kidneys, etc. would be helpful in further characterizing the peptide. Suitable in vitro and in vivo assays are disclosed in U.S. patent application Ser. Nos. 10/188,905 and 10/185,841, each of which is incorporated by reference herein in its entirety. 7.2 Assays for Specific Indications

A. Toxic Agents.

The isolated peptides to be used within the method of the current invention may be demonstrated to inhibit damage, effects or symptoms resulting from exposure to a toxic agent in vitro or in vivo using a variety of assays known in the art, or described herein.

Further peptides used within the method of the Invention, may be tested in various in vitro assays in the art to determine their ability to prevent, treat, ameliorate, or manage damage, effects or symptoms resulting from exposure to a toxic agent. In general, this is accomplished by selecting an appropriate cell line, subjecting that cell to a toxic agent of interest and treating a portion of the cells with a peptide of interest and determining the cells survival or response in the presence of the toxic agent and in the presence of the toxic agent and the peptide of interest. If the cell exhibits improved survival or a reduction of damage, effects or symptoms in the presence of the peptide, the peptide can be considered to he a possible therapeutic for toxic exposure. Further one of ordinary skill in the art will recognize that the peptides ability as a protectant can be evaluated by treating the cells with the peptide prior to the toxic agent challenge.

For example, suitable assays for toxic agents include, but arc not limited to: Chemical Agents: a) skin cell lines such as J-774 (mouse macrophage derived cell line), CHO-K1 (strain of epithelial cell line derived from Chinese hamster ovary cells), and HeLa (human cervical carcinoma) (Sawyer, T. et al., Hypothermia as an adjunct to vesicant-induced skin injury, Eplasty 2008; 8:e25); b) corneal cell lines for vesicant agents (Amir, A. et al, The corneal epithelium in sulfur mustard ocular injury—In vitro and ex vivo studies, Proceedings of the U.S. Army Medical Defense Bioscience Review, Aberdeen Proving Ground, Md. (2004)); e) macrophages (Amir A., et al., Sulfur mustard toxicity in macrophages; effect of dexamethasone, J Appl Toxicol, 20 Suppl 1:S51-8 (2000)); d) upper respiratory tract cell lines (Andrew, D. J. and C. D. Lindsay, Protection of human upper respiratory tract cell lines against sulphur mustard toxicity by gluthione esters, Hum Exp Toxicol 17(7):387-95 (1998); Calvet et al., Airway epithelial damage and release of inflammatory mediators in human lung parenchyma after sulfur mustard exposure, Hum Exp Toxicol 18(2):77-81(1999); Langford, A. M. et al., The effect of sulphur mustard on glutathione levels in rat lung slices and the influence of treatment with arylthiols and cysteine esters, Hum Exp Toxicol 15(8):619-24); e) skin models (Bl aha et al., Effects of CEES on inflammatory mediators, heat shock protein 70A, histology and ultrastructure in two skin models, J Appl Toxicol 20 Suppl 1:S101-8 (2000); Henemyre-Harris et al., An in vitro wound healing model to screen pharmacological interventions for the effective treatment of cutaneous sulfur mustard injuries, Proceedings of the U.S. Army Medical Defense Bioscience Review, Aberdeen Proving Ground, Md. (2004))(See generally, www.counteract.rutgers.edu/invitro.html for additional literature on appropriate in vitro studies); Radiation Agents: a) endothelial cells (Abderrahmani, R, et al., Role of plasminogen activator inhibitor type-1 in radiation-induced endothelial cell apoptosis, Radioprotection 2008, vol 43, no. 5. b) neuroimmune cells (afferent nerves, enteric sensory nerves, mast cells) (Wang, J. et al., Neuroimmune interactions: potential target for mitigating or treating intestinal radiation injury, British Journal of Radiology (2007) 80, S41-548), c) blood or lymphocyte cultures (Lloyd D C et al., Phys Med Biol 18(3):421-31 (1973); Lloyd D C et al., Mutat. Res. 179(2):197-208 (1987); Blakely W F et al., Stem Cells 13 (Suppl 1):223-30 (1995): Gotoh E et al., Int. J. Radiation. Biol. 81(1):33-40 (2005)); Biological Agents: (a) peripheral blood mononuclear cells (Rasha, H. et al. Modeling of SEB-induced host gene expression to correlate in vitro to in vivo responses: Microarrays for biodefense and environmental applications, Biosensors and Bioelectrics (2004) vol. 20, no. 4, 719-727).

Further, suitable in vivo assays are known in the art for evaluating the effect of therapeutics on toxic agent exposure. Animal models using rats, mice, guinea pigs, rabbits, pigs, sheep, ferrets, dogs and non-human primates are contemplated as well as transgenic animals that are particularly susceptible to a toxic agent (CD46 mice). In particular, assays known in the art include, but are not limited to: Chemical Agents: (1) Reid, F. M., Sulfur mustard induced skin burns in weanling swine evaluated clinically and histopathologically, Journal of applied toxicology, vol. 20 (S1), pages S153-S160 (2001); (2) Isidore, M. A. et al.. A dorsal model for cutaneous vesicant injury 2-chloroethyl ethyl sulfide using c57b1/6 mice, Cutaneous and ocular toxicology, Vol. 26 (3), 265-276 (2007); (3) See generally, www.counteract.rutgers.edu/animal.html; (4) Kassa J., et. al., The Choice: H1-6, pradoxime or Obidoxime against Nerve Agents?, www.asanite.com/ASANews-97/Antidot-Choice.html, (5) Shill, T M et al., Organophosphorus nerve agents-induced seizures and efficacy of atropine sulfate as anticonvulsant treatment, Pharmacol-Biochem-Behav. 1999 September, 64(1), 147-53, (6) Luo, C et al., Comparison of oxime reactivation and aging of the nerve agent-inhibited monkey and human acetylcholinestemses, Chemico-Blological Interactions, 175(1-3), 261-266 (2008); Radiation Agents: (1) W. F. Blakely et al., In Vitro and Animal Models of Partial-Body Dose Exposure: Use of Cytogenic and Molecular Biomarkers for Assesment of Inhomogeneous Dose Exposures and Radiation Injury, PB-Rad-lnjury 2008 Workshop, May 5-6, 2008 AFRRI, Bethesda, Md.; (2) Augustine, A et al., Meeting Report: Animal Models of Radiation Injury, Protection and Therapy, Radiation Research 164: 100-109 (2005); (3) Houchen, C et al. Prosurvival and antiapoptotic effects of PGE₂ in radiation injury are mediated by EP₂ receptor in intestine, Am J Physiol Gastrointest Liver Physiol, 284: G490-G498, 2003; (4) Jichun Chen, Animal Models for Acquired Bone Marrow Failure Syndromes, Clinical Medicine & Research 3(2): 102-108: Biological Agents: (1) Biodefense: Research Methodology and Animal Models, James R, Swearengen (editor) 2006 CRC Press.

B. Inflammation

Additionally, various in vitro models of inflammation may be used to evaluate a peptides ability to protect or treat the damage, symptoms, or effects of inflammation on the body. Initially, the ability of the peptide to modulate an inflammatory mediator can be confirmed by measuring the levels of the inflammatory mediator in an inflammatory assay after treatment with the peptide by known methods, including but not limited to, ELISA, cytometric bead array analysis, high-sensitivity and immunonephelometric assays. For example to determine if the peptide modulates either TNF-α or IL-1, a murine model of LPS-mediated cytokine production would be performed. Some mice in the murine model would he pretreated with the peptide of interest and then challenged with LPS while others would be saline treated. Blood would then be collected and the TNF-α and IL-1 levels in the blood could be determined by an ELISA kit (OPT-EIA mouse TNF-α and IL-1 ELISA kits (BD Biosciences). If the TNF-α levels in the treated animals arc lower than the TNIF-α levels in the saline treated animals then the peptide could be considered to modulate TNF-α. Preferably, the peptide would he tested for its ability to modulate more than one inflammatory mediator, and more preferably it would he a mediator other than or in addition to TNF-α, and most preferably it would be histamine. Similarly, the peptides may be tested in additional in vitro assays including, but not limited. to, those disclosed in Lopata, Andreas L., Specialized in vitro Diagnostic Methods In. The Evaluation Of Hypersensitivit—An Overview, Current Allergy & Clinical Immunology, March 2006, Vol. 19, No. 1, (histamine and tryptase assays), and Arulmozhi et al., Pharmacological Investigations of Sapindus trifoliatus in various in vitro and in vivo models of inflammation, Indian Journal of Pharmacology, vol. 37:2, 96-102 (2005) (5-lipoxygenase (5-LO), cyclo-oxygenase (COX), Leukotrine B4 (LTB4) and nitric oxide synthase (NOS)).

Further, in vivo assays of inflammation may be useful in evaluating the peptides utility as a therapeutic against toxic agents. In vivo assays, including, but not limited to, murine EAE models, those utilizing transgenic mice such as MDBiosciences DSS IBD murine model of severe colitis, the MDBioscience TNBS IBD murine model of inflammatory bowel disease, models involving IL-1 knockout mice disclosed within U.S. Pat. No. 6,437,216, or models of transgenic mice involving TNF-α as disclosed within Probert et al. Spontaneous inflammatory demyelinating disease in transgenic mice showing CNS-specific expression of tumor necrosis factor α. Proc. Natl. Acad. Sci. 1995 USA 92, 11294-11298, Kontoyiannis et al, Impaired on/off regulation of TNF biosynthesis in mice lacking TNF AU-rich elements: implications for joint and gut-associated immunopathologies, Immunity 10:387-398, 1999, Keifer et al. Transgenic mice expressing human tumour necrosis factor: a predictive genetic model of arthritis. EMBO J. 1991 December; 10(13):4025-31, or models using chemical or synthetic challenges to induce the inflammation such as models of asthma and chronic obstructive pulmonary disease disclosed in JPET 307:373-385, 2003, adjuvant arthritis models as disclosed in EP 1 777 234; murine LPS shock models, murine LPS lung models, acute paw inflammation models, or histidine challenge wheal formation model as disclosed in detail below.

Further, the efficacy of the compounds in humans using well-known clinical studies such as the skin prick test and bronchoprovocation test disclosed in Ravensberg et al. “Validated safety predictions of airway responses to house dust mites in asthma,” Clinical and Experiment & Allergy, 37:100-107 (2007); asthma studies as disclosed in Diamant et al. “Methods used in clinical development of novel anti-asthma therapies,” Respiratory Medicine (2008) 102, 332-338, or nasal allergen challenge as disclosed in Boot et al. “Nasal Nitric Oxide: longitudinal reproducibility and the effects of a nasal allergen challenge in patients with allergic rhinitis,” Allergy 2007:62 :378-384.

Cancer.

The isolated peptides to he used within the method of the current invention may be demonstrated to inhibit tumor cell proliferation, cell transfomation and tumorigenesis in vitro or in vivo using a variety of assays known in the art, or described herein. Such assays can use cells of a cancer cell line or cells from a patient. Many assays well-known in the art can be used to assess such survival and/or growth; for example, cell proliferation can be assayed by measuring ³H-thymidine incorporation, by direct cell count, by detecting changes in transcription, translation or activity of known genes such as prow-oncogenes (e.g., fos, myc) or cell cycle markers (Rb, cdc2, cyclin A, D1, D2, D3 or E). The levels of such protein and mRNA and activity can be determined by any method well known in the art. For example, protein can be quantitated by known immunodiagnostie methods such as Western blotting or immunoprecipitation using commercially available antibodies (for example, many cell cycle marker antibodies are from Santa Cruz, Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art. Differentiation can be assessed visually based on changes in morphology, etc.

The present invention provides for cell cycle and cell proliferation analysis by a variety of techniques known in the art, including but not limited to the following:

As one example, bromodeoxyuridine (“BRDU”) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38, 369; Campana et at., 1988, J. Immunol. Meth. 107, 79).

Cell proliferation may also he examined using (³H)-thymidine incorporation (see e.g., Chen, J., 1996, Oneogene 13:1395 403; Jeoung, J., 1995, J. Biol. Chem. 270:18367 73). This assay allows for quantitative characterization of S-phase DNA synthesis. In this assay, cells synthesizing DNA will incorporate ³H-thyrnidine into newly synthesized DNA. Incorporation may then be measured by standard techniques in the art such as by counting of radioisotope in a Scintillation counter (e.g., Beekman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189 199; Vassilev et al., 1995, J. Cell Sci. 108:1205 15).

Cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum. iodide assay (see e.g., Turner, T., et al., 1998, Prostate 34:175 81). Alternatively, the DNA ploidy may be determined by quantitation of DNA Feulgen staining (which binds to DNA in a stoichiometric manner) on a computerized microdensitometrystaining system (see e.g., Bacus, S., 1989, Am. J. Pathol. 135:783 92). In another embodiment, DNA content may be analyzed by preparation of a chromosomal. spread (Zabalou, S., 1994, Hereditas. 120:127 40; Pardue, 1994, Meth. Biol. 44:333 351),

The expression of cell-cycle proteins (e.g., CycA, CycB, CycE, CycD, cdc2, Cdk4/6, Rb, p21 or p27) provide crucial information relating to the proliferative state of a cell or population of cells. For example, identification in an anti-proliferation signaling pathway may be indicated by the induction of p21cip1. Increased levels of p21 expression in cells results in delayed entry into G1 of the cell cycle (Harper et al., 1993, Cell 75:805 816; Li et at., 1996, Curr. Biol. 6:189 199). p21 induction may be identified by immunostaining using a specific anti-p21 antibody available commercially (e.g., from Santa Cruz, Inc.). Similarly, cell-cycle proteins may be examined by Western blot analysis using commercially available antibodies. In another embodiment, cell populations are synchronized prior to detection of a cell cycle protein. Cell-cycle proteins may also be detected by FACS (fluorescence-activated cell sorter) analysis using antibodies against the protein of interest.

Detection of changes in length of the cell cycle or speed of cell cycle may also be S used to measure inhibition of cell proliferation by a peptide of the Invention. In one embodiment the length of the cell cycle is determined by the doubling time of a population of cells (e.g., using cells contacted or not contacted with one or more peptide of the Invention). In another embodiment, FACS analysis is used to analyze the phase of cell cycle progression, or purify G1, S, and G2/M fractions (see e.g., Delia, D. et al., 1997, Oncogene 14:2137 47).

Lapse of cell cycle checkpoint(s), and/or induction of cell cycle checkpoint(s), may be examined by the methods described herein, or by any method known in the art. Without limitation, a cell cycle checkpoint is a mechanism which ensures that a certain cellular events occur in a particular order. Checkpoint genes arc defined by mutations that allow late events to Occur without prior completion of an early event (Weinert, T., and Hartwell, L., 1993. Genetics, 134;63 80). induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g. progression into mitosis without complete replication of the genomic DNA).

In addition to the effects of expression of a particular cell cycle protein, activity and post-translational modifications of proteins involved in the cell cycle can play an integral role in the regulation and proliferative state of a cell. The invention provides for assays involved detected post-translational modifications (e.g., phosphorylation) by any method known in the art. For example, antibodies that detect phosphorylated tyrosine residues are commercially available, and may be used in Western blot analysis to detect proteins with such modifications. In another example, modifications such as myristylation, may be detected on thin layer chromatography or reverse phase h.p.l.c. (see e.g. Glover, C., 1988. Biochem. J. 250:485 91; Paige, L., 1988, Biochem J.; 250:485 91).

Activity of signaling and cell cycle proteins and/or protein complexes is often mediated by a kinase activity. The present invention provides for analysis of kinase activity by assays such as the histone H1 assay (see e.g., Delia, D. et al., 1997, Oneogene 14:213747).

The peptides used within the method of the invention can also be demonstrated to alter cell proliferation in cultured cells in vitro using methods which are well known in the art. Specific examples of cell culture models include, but are not limited to, for lung cancer, primary rat lung tumor cells (Swafford et al., 1997, Mol. Cell. Biol, 17:1366 1374) and large-cell undifferentiated cancer cell lines (Mabry et al., 1991, Cancer Cells, 3:53 58); colorectal cell lines for colon cancer (Park and Gazdar, 1996, J. Cell Biochem, Suppl. 24:131. 141); multiple established cell lines for breast cancer (Hambly et al., 1997, Breast Cancer Res. Treat, 43:247 258; Gierthy et aL, 1997, Chemo,sphere 34:1495 1505; Prasad and Church, 1997, Biochem. Biophys. Res. Commun. 232:14 19); a number of well-characterized cell models for prostate cancer (Webber et al., 1996, Prostate, Part 1, 29:386 394; Part 2, 30:58 64; and Part 3, 30:136 142; Boulikas, 1997, Anticancer Res. 17:1471 1505); for genitourinary cancers, continuous human bladder cancer cell lines (Ribeiro et al., 1997, Int. Radiat. Biol. 72:11 20); organ cultures of transitional cell carcinomas (Booth et al., 1997, Lab Invest. 76:843 857) and rat progression models (Vet et al., 1997, Biochim. Biophys Acta 1360:39 44); and established cell lines for leukemias and lymphomas (Drexler, 1994, Leuk. Res. 18:919 927, Tohyama, 1997, Int. J. Hematol. 65:309 317).

The peptides of the Invention can also be demonstrated to inhibit cell transformation (or progression to malignant phenotype) in vitro, in this embodiment, cells with a transformed cell phenotype are contacted with one or more peptides of the Invention, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc, (see Luria et al., 1978, General Virology, 3d Ed., John Wiley & Sons, New York, pp. 436446),

Loss of invasiveness or decreased adhesion. may also be used to demonstrate the anti-cancer effects of the peptides used in the method of the Invention. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness may be measured by a variety of techniques known in the art includina, for example, induction of E-cadherin-mediated cell-cell adhesion. Such E-cadherin-mediated adhesion can result in phenotypic reversion and loss of invasiveness (Hordijk et al., 1997, Science 278:1464 66).

Loss of invasiveness may further be examined by inhibition of cell migration. A variety of 2-dimensional and 3-dimensional cellular matrices are commercially available (Calbiochem-Novabiochcm Corp. San Diego, Calif.). Cell migration across or into a matrix may be examined by microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., 1997, Science 278:1464 66).

Alternatively, loss of invasiveness may be measured by cell migration through a chemotaxis chamber (Neuroprobe/Precision Biochemicals Inc. Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated may then be correlated with invasiveness (see e.g., Ohrtishi, T., 1993, Biochem. Biophys. Res. Commun. 193:518 25).

The peptides used in the method of the Invention can also be demonstrated to inhibit tumor formation in vivo. A vast number of animal models of hyperproliferative disorders, including tumorigenesis and metastatic spread, are known in the art (see Table 317-1, Chapter 317, “Principles of Neoplasia,” in Harrison's Principles of Internal Medicine, 13th Edition, Isselbacher et al., eds., McGraw-Hill, p. 1814, and Lovejoy et al., 1997, J. Pathol. 181:130 135). Specific examples include for lung cancer transplantation of tumor nodules into rats (Wang et al 1997 Ann. Thorac. Surg. 64:216 219) or establishment of lung cancer metastases in SCID mice depleted of NK cells (Yong and Sone, .1997, Gan To Kagaku Ryoho 24:489 494); for colon cancer, colon cancer transplantation of human colon cancer cells into nude mice (Gutman and Fidler, 1995, World J. Surg, 19:226 234), the cotton top tamarin model of human ulcerative colitis (Warren, 1996, Aliment. Pharmacal. Thep. 10 Supp 12:45 47) and mouse models with mutations of the adenomatous polyposis tumor suppressor (Polak is, 1997, Biochim. Biophys. Acta 1332:F127 F147); for breast cancer, transgenic models of breast cancer (Dankort and Muller, 1996, Cancer Treat. Res. 83:71 88; Amundadittir et al., 1996, Breast Cancer Res. Treat, 39:119 135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35 40); for genitourinary cancers, induced bladder neoplasm in rats and mice (Oyasu, 1995, Food Chem. Toxicol 33:747 755) and xenografts of human transitional cell carcinomas into nude rats (Jarrett et al., 1995, J. Endourol. 9;1 7); and for hcmatopoietic cancers, transplanted allogeneic marrow in animals (Appelbaum, 1997, Leukemia 11 (Suppl. 4):S15 S17). Further, general animal models applicable to many types of cancer have been described, including, but not restricted to, the p53-deficient mouse model (Donehower, 1996, Semin. Cancer Biol. 7:269 278), the Min mouse (Shoemaker et al., 1997, Biochem. Biophys. Acta, 1332:F25 F48), and immune responses to tumors in rat (Frey, 1997, Methods, 12:173 188).

For example, a peptide to be used in the method of the Invention can be administered to a test animal, in one embodiment a test animal predisposed to develop a type of tumor, and the test animal subsequently examined for a decreased incidence of tumor formation in comparison with an animal not administered the peptide of the Invention. Alternatively, a peptide of the Invention can be administered to test animals having tumors (e.g., animals in which tumors have been induced by introduction of malignant, neoplastic, or transformed cells, or by administration of a carcinogen) and subsequently examining the tumors in the test animals for tumor regression in comparison to animals not administered the peptide of the invention.

8. Therapeutic Use

A. Modulation of Mediators of the Body's Response

One of ordinary skill in the art would recognize that the peptides of the current invention may be used to modulate the effects of the body's response to a disease or disorder associated with tissue damage. In particular, one example of mediators the peptides noted above may be used to modulate are inflammatory modulators, including but not limited to, plasma derived inflammatory mediators, such as bradykinins, C3, C5a, Factor XII, membrane attack complex, Hageman factor, plasmin, thrombin, lymphokines (macrophage activating factor (MAF), macrophage migration inhibition factor (MMIF), macrophage chemotactic factor (MCF), leukocyte migration inhibition factor (LMIF), histamine releasing factors (HRFs), and transfer factor (TF)); interleukins (IL-1L-2, IL-3, IL-4, . . . IL-15); Tumor necmsis factors (TNF-α (cachectin), TNF-β (lymphotoxin)); interferons (IFN-α, IFN-β, IFN-γ, IFN-ω, IFNτ); Colony stimulating factors (granulocyte colony stimulating factor (G-CSF), granulocyte-macrophage colony stimulating factor (GM-CSF), macrophage colony stimulating factor (M-CSF), and multi colony stimulating factor (IL-3)); polypeptide growth factors (acidic fibroblast growth factor (aFGF), basic fibroblast growth factor (bFGF), epidermal growth factor (EGF); nerve growth factor (NGF), platelet-derived growth factor (PDGF), and vascular endothelial growth factor (VEGF)); Transforming growth factors (TGF-α and TGE-β), α-Chemokines (IL-8, neutrophil-activating protein 2 (NAP-2), platelet factor-4 (PF-4), and β-throboglobulin (βTG)); β-Chemokines (monocyte chemoattractant protein 1 (MCP-1), MCP-3, MIP-1α, macrophage inflammatory protein 1β (MIP-1β), regulated upon activation normal T expressed and presumably secreted ehemnokine (RANTES)) and Stress proteins (heat shock proteins (HSPs), glucose related proteins (GSPs), ubiquitin, and superoxide dismutase (Mn)), leukemia inhibitory factor (LIF), oncostatin (OSM), ciliary neurotrophic factor (CNTF), platelet basic protein (PBP), lysosome granules, histamine, serotonin, leukotriene B4, nitric oxide, and/or prostaglandins. In a preferred embodiment the peptides inhibit or surpress the activity of the mediators and more preferably inhibit the activity of TNF-α, histamine, nitric oxide, and interleukins. Most preferably, the peptides inhibit the activity of two or more inflammatory mediators.

B. Treatment or Prevention of Various Diseases, Disorders, and Conditions

The tissue protective peptides and peptide analogs of the current invention are also useful as therapeutics for treatment or prevention of various diseases, disorders, and conditions. One skilled in the art would also recognize that such peptides and peptide analogs can be used to achieve modulation of a tissue protective receptor complex, e.g., tissue protective cytokine complex. Both in vitro and in vivo techniques that can be used for assessing the therapeutic indications of, for example, the compounds identified by the inventive assays disclosed above are disclosed in PCT Application No. PCT/US01/49479, U.S. patent application Ser. Nos. 10/188,905 and 10/185,841.

The aforementioned tissue protective peptides and peptide analogs of the invention may be useful generally for the prevention, therapeutic treatment, or prophylactic. treatment of human diseases or disorders of the central nervous system or peripheral nervous system which have primarily neurological or psychiatric symptoms, ophthalmic diseases, cardiovascular disuses, cardiopulmonary diseases, respiratory diseases, kidney, urinary and reproductive diseases, bone diseases, skin diseases, connective tissue diseases, gastrointestinal diseases and endocrine and metabolic abnormalities. Examples of use include, but are not limited to, protection against and repair of injury resulting from trauma and resulting inflammation to the brain (ischemic stroke, blunt trauma, subaracfmoid hemorrhage), spinal cord (ischemia, blunt force trauma), peripheral nerves (sciatic nerve injury, diabetic neuropathy, carpal tunnel syndrome), retinal (macular edema, diabetic retinopathy, glaucoma), and heart (myocardial infarct, chronic heart failure), in particular, such diseases, disorders, and conditions include hypoxic conditions, which adversely affect responsive tissues, such as excitable tissues including, but not limited to, those noted above in Section 4.2 (xiii), or those responsive cells tissues or organs, those that express the appropriate Type-1 cytokinc receptor, e.g., EPO-R receptor or the tissue protective receptor complex. Therefore, the tissue protective peptides and peptide analogs of the invention can be used to treat or prevent damage to responsive tissue resulting from hypoxic conditions in a variety of conditions and circumstances. Non-limiting examples of such conditions and circumstances are provided in the table herein below.

The tissue protective peptides and peptide analogs are also of interest in the modulation of stern cell activity. It has been established that cytokines exhibiting tissue protective activity, e.g. EPO, are able to mobilize stem cells, stimulating the migration to regions of injury and aiding the repair process, e.g. in a regenerative role. For example, in experimental stroke, EPO mediates the migration of neuroblasts into a region of ischemic injury to regenerate neurons during the period of recovery (Tsai et al. J. Neurosci (2006) 26:1269-74). As another example, EPO and carbamylated EPO (CEPO) mobilize endothelial progenitor cells from the bone marrow into the circulation. These cells then home to distance regions and arc involved in the formation of new blood vessels (for effect of EPO, see, Bahlmann et al, 2003, Kidney Int. 64:1648-1652). While not wishing to be bound to any particular theory, the isolated peptides and peptide analogs disclosed herein are believed to have a similar effect on the migration of stem cells.

In the example of the protection of neuronal tissue pathologies treatable and preventable using tissue protective peptides and peptide analogs of the invention, such pathologies include those which result from reduced oxygenation of neuronal tissues. Any condition which reduces the availability of oxygen to neuronal tissue, resulting in stress, damage, and finally, neuronal cell death, can be treated using tissue protective peptides and peptide analogs of the present invention. Generally referred to as hypoxia andlor isehemia, these conditions arise from or include, but are not limited to, stroke, vascular occlusion, prenatal or postnatal oxygen deprivation, suffocation, choking, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, sickle cell crisis, cardiac arrest, dysrhythmia, nitrogen narcosis, hypoxemic hypoxia (altitude sickness, high altitude pulmonary edema, high altitude cerebral edema, sleep apnea, hypopnea, respiratory arrest, shunts), methaemoglobinaemia, histotoxic hypoxia, intrauterine hypoxia, and neurological deficits caused by heart-lung bypass procedures.

In one embodiment, for example, the tissue protective peptides and peptide analogs of the present invention identified using the above-noted assays could be administered alone or as part of a composition to prevent injury or tissue damage resulting from risk of injury or tissue damage prior to, during, or subsequent to a surgical procedure or a medical procedure. For example, surgical procedures may include tumor resection or aneurysm repair and medical procedures may include labor or delivery. Other pathologies caused by or resulting from hypoglycemia which are treatable using tissue protective peptides and peptide analogs of the present invention include insulin overdose, also referred to as iatrogenic hyperinsulinemia, insulinoma, growth hormone deficiency, hypocortisolism, drug overdose, and certain tumors.

Other pathologies resulting from excitable neuronal tissue damage include seizure disorders, such as epilepsy, convulsions, or chronic seizure disorders. Other treatable conditions and diseases include, but are not limited to, diseases such as stroke, multiple sclerosis, hypotension, cardiac arrest, chronic heart failure, Alzheimer's disease, Parkinson's disease, cerebral palsy, brain or spinal cord trauma, AIDS dementia, age-related loss of cognitive function, memory loss, amyotrophic lateral sclerosis, seizure disorders, alcoholism, retinal ischemia, optic nerve damage resulting from glaucoma, and neuronal loss.

The specific tissue protective peptides and peptide analogs of the present invention may be used to treat or prevent inflammation resulting from disease conditions or various traumas, such as physically or chemically induced inflammation. The tissue protective peptides and peptide analogs are also contemplated for the treatment and prevention of inflammatory conditions in one or more organs or tissues including, but not limited to, the brain, spinal cord, connective tissue, heart, lung, kidney and urinary tract, pancreas, eyes and prostate. Non-limiting examples of such trauma include, but are not limited to those listed in Section 4.2 (xvi). Further the tissue protective peptides may used to treat or prevent inflammation resulting from ischemic and non-ischemic conditions including, but not limited to, allergies, allergic diseases, allergic symptoms, rheumatic diseases, sports related injuries, exposure to toxic agents, infections including viral, fungal, and bacterial, further examples of such conditions are disclosed above in Section 4.2(iv), (v) and (xvi). The inflammation may be acute or chronic. Further applications in the field of inflammation are noted within. PCT/US2004/031789 filed Sep. 29, 2004 and published as WO 2005/032467.

The specific tissue protective peptides and peptide analogs of the present invention may be used to treat central nervous and peripheral nervous system diseases resulting from demyelination or impairment of the myelin sheath. These diseases are defined as mainly involving inflammatory myelin sheath lesions of unknown origin, with the exception of myelination deficiency diseases, such as lenkodystrophy, and diseases due to obvious causes. Multiple sclerosis (MS) is a typical disease among demyelinating diseases, and pathologically, it is characterized by changes, mainly, inflammatory demyelination, and gliosis. Since its etiology is unknown, its diagnosis is made based on its clinical features, i.e., spatial multiplicity and multiplicity over time of central nervous system lesions. Furthermore, acute disseminated encephalomyelitis (ADEM), inflammatory diffuse sclerosis, acute and subacute necrotizing hemorrhagic encephalomyelitis, and transverse myelitis are included in demyelinating diseases. Also, peripheral nervous tissues rely upon Schwann cells to maintain the myelin sheath, if these cells are impaired, peripheral demyelinating disease is caused.

The tissue protective peptides and peptide analogs of the present invention may be used to treat or prevent conditions of, and damage to the heart including any chronic or acute pathological event involving the heart and/or associated tissue (e.g., the pericardium, aorta and other associated blood vessels), including ischemia-reperfusion injury; congestive heart failure; cardiac arrest; myocardial infarction; atherosclerosis, mitral valve leakage, atrial flutter, cardiotoxicity caused by compounds such as drugs (e.g., doxorubicin, herceptin, thioridazine and cisapride); cardiac damage due to parasitic infection (bacteria, fungi, rickettsiae, and viruses, e.g., syphilis, chronic Trypanosoma cruzi infection); fulminant cardiac arnyloidosis; heart surgery; heart transplantation; angioplasty, laparoscopic surgery, traumatic cardiac. injury (e.g., penetrating or blunt cardiac injury, and aortic valve rupture), surgical repair of a thoracic aortic aneurysm; a suprarenal aortic aneurysm; cardiogenic shock due to myocardial infarction or cardiac failure; neurogenic shock and anaphylaxis. The tissue protective peptides and peptide analogs of the current invention may also be used to treat those individuals at risk for heart disease such as cardiac failure (i.e., where the heart is not able to pump blood at a rate required by the metabolizing tissues, or when the heart can do so only with an elevated filling pressure). Such at risk patients would include patients having or being at risk of having cardiac infarction, coronary artery disease, inyocarditis, chemotherapy, cardiomyopathy, hypertension, valvular heart diseases (most often mitral insufficiency and aortic stenosis) and toxin-induced cardiomyopathy (e.g. ethanol, cocaine, etc.) and the like.

The tissue protective peptides and peptide analogs of the present invention may be used to treat or prevent conditions of, and damage to, the eyes, e.g., retinal tissue. Such disorders include, but are not limited to retinal ischemia, macular degeneration, retinal detachment, retinitis pigmentosa, arteriosclerotic retinopathy, hypertensive retinopathy, retinal artery blockage, retinal vein blockage, retinal edema, hypotension, and diabetic retinopathy.

In another embodiment, the tissue protective peptides and peptide analogs of the present invention and principles of the invention may be used. to prevent or treat injury resulting from exposure to toxic agents, i.e. radiation or chemical damage to responsive tissue. In one embodiment of the invention the above-noted peptides are useful as therapeutics for modulating the mediators of the body's response to toxic agents, preferably to suppress or inhibit the activity of such modulators. Additionally, the above-noted peptides are useful as therapeutics for the treatment, prevention, amelioration or management of damage, effects or symptoms of exposure to a toxic agent. The peptides may be used to treat exposure to various toxic agents, including biological, chemical or radiation agents.

These peptides may be used to treat the damages, effects, or symptoms due to biological agents such as of prions, viruses, microorganisms (bacteria and fungi), and some unicellular and multicellular eukaryotes (i.e., parasites), including, but not limited to, those biological toxins listed above in Section 4.2 (viii). Further the peptides of the current invention may be used to prevent, treat, ameliorate, or manage the damage, effects or symptoms due to chemical agents. Such agents include, but are not limited to, blood agents, blister agents, nerve agents, pulmonary agents, and incapacitating agents. Additionally, the peptides of the current invention may be used to prevent, treat, ameliorate or manage damage, effects or symptoms due to toxic exposure to industrial chemicals including but not limited to those listed in Section 4.2 (x). Damage, effects or symptoms due to exposure to a radiation agent are preventable, treatable, or manageable using the peptides of the current invention. The peptides can prevent, treat, ameliorate, or manage the damage, effects or symptoms due to radioactive agents that include alpha, beta or gamma radiation, and more particularly may include, but are not limited to, ¹³⁷Cs, ⁶⁰Co, ²⁴¹Am, ²⁵²Cf, ¹⁹²Ir, ²³⁸Pu, ⁹⁰Sr, ²²⁶Ra, ⁹¹Sr, ⁹²Sr, ⁹⁵Zr, ⁹⁹Mo, ¹⁰⁶Ru, ¹³¹Sb, ¹³²Te, ¹³⁹Te, ¹⁴⁰Ba, ¹⁴¹La, ¹⁴⁴Ce, ²³³U, ²³⁵U, ²³⁸U, ²²⁸P, ²²⁹P, ²³⁰P, ²³¹P, ²³²O, ²³³P, ²³⁴P, ²³⁵P, ²³⁶P, ²³⁷P, ²³⁸P, ²⁴⁹P, ²⁴⁰P, ²⁴¹P, ²⁴²P, ²⁴³P, ²⁴⁴P, ²⁴⁵P, ²⁴⁶P, ²¹⁷P, and ¹³¹I. Further, one of ordinaly skill in the art will recognize that the peptides may also be used to prevent, mediate, treat or ameliorate the damages, effects or symptoms due to the cumulative or synergistic use of these toxic agents (i.e., the use of a radioactive agent prior to dispersing a biological agent so that the victim's will be more susceptible to the biological agent, administering a vesicant agent in conjunction with a nerve agent to prevent the victims from effectively seeking refuge or aid, tainting bullets or shrapnel with biological or radioactive agents to inhibit or complicate the healing process, etc.) Preferably, peptides of the current invention will be able to treat, mediate, ameliorate or prevent toxic effects on several different types of cells, organs, or tissues for example in two or more of the following central nervous, peripheral nervous, ophthalmic, cardiovascular, cardiopulmonary, respiratory, kidney, urinary, reproductive, musculoskeletal, skin, connective tissue, gastrointestinal, hematopoietic, endocrine, and metabolic. Further, a peptide of the current invention would be effective as a therapeutic or preventive for more than one toxic agent within the same class (i.e., against more than one type of chemical, biological or radioactive agent—a preventive against a vesicant and nerve agents for example) or different classes of toxic agents (i.e. a therapeutic for exposure to a radioactive agent and a chemical agent). A further utility of the tissue protective peptides and peptide analogs of the present invention is in the treatment of poisoning, such as neurotoxin poisoning (e.g., domoic acid shellfish poisoning), toxins (ethanol, cocaine, etc.), as the result of chemotherapeutic agents of radiation exposure; neumlathyrism; Guam disease; amyotrophic lateral sclerosis; and Parkinson's disease.

As mentioned above, the present invention also provides tissue protective peptides and peptide analogs of the present invention for use in enhancing tissue function in responsive cells, tissues and organs in a mammal by peripheral administration of a tissue protective peptide as described above. Various diseases and conditions are amenable to treatment using this method. For example this method is useful for enhancing function in excitable tissues resulting in an increase in cognitive function even in the absence of any condition or disease. Further, the tissue protective cytokines are useful for improving the quality of wound healing, reducing the time required to heal, improving the quality of the healed tissues and reducing the incidence of adhesions resulting from the wound. See PCT/US2004/031789 filed Sep. 29, 2004 and published as WO 2005/032467. Further the tissue protective peptides of the current invention may be useful in treating, preventing or managing the lesions on the skin or along the respiratory pathways induced by chemical agents such as blistering or vessicant agents or industrial chemicals.

These uses of the peptides of the present invention are describe in further detail below and include enhancement of learning and training in both human and nonhuman mammals.

In another embodiment, the tissue protective peptides and peptide analogs of the present invention may be useful generally for the prevention, therapeutic treatment, prophylactic treatment or management of various cancers or neoplastic disorders of the central nervous system, peripheral nervous system, gastrointestinal/digestive system, genitourinary system, adrenal, gynecological, head and neck, hematological/blood, musculoskeletal/soft tissue, respiratory, and breast. Examples of use include, but are not limited to, protection against and repair of it resulting from cancers or neoplastic disorders listed in section 4.2(ix) and (xxv). Further the peptides of the current invention may be used for the prevention, therapeutic treatment, prophylactic treatment or management of various syndromes associated with neoplasms or cancers, including, but not limited to those listed above in Section 4.2 (xxviii). The peptides may be used in accordance with the method of the current invention to address the above-noted syndromes. For example, the peptides may be administered to address hereditary syndromes such as Li Fraurneni, hereditary nonpolyposis colorectal cancer, familial adenomatous polyposis, and Min Hippel-Lindau syndrome by either delaying the onset of the neoplastic aspects of the disease, reducing the number of neoplastic growths associated with the syndrome, or in general enhancing the quality of life or the longevity of those patients afflicted with these conditions. The peptides may also be administered prophylactically to address syndromes related to certain treatment, chemotherapy or radiation therapy, of the neoplastic disorder or cancer, such as androgen deprivation syndrome, therapy related myelodysplastic syndrome or somnolence syndrome, in the hopes of preventing the syndromes or reducing the severity of the syndrome.

Further, the peptides may be used to treat or prevent cachexia and diseases related to cachexia. Such diseases include, but are not limited to cancer cachexia, anorexia, asthenia, anemia, tuberculosis, AIDS, congestive heart failure, renal failure, liver failure, chronic Obstructive pulmonary disease, emphysema, muscle atrophy, diabetes, and endotoxinemia.

Conditions and diseases treatable or preventable using tissue protective peptides and peptide analogs of the present invention provides the central nervous system include but are not limited to mood disorders, anxiety disorders, depression, autism, attention deficit hyperactivity disorder, and cognitive dysfunction. These conditions benefit from enhancement of neuronal function. Other disorders treatable in accordance with the teachings of the present invention include sleep disruption, for example, sleep apnea and travel-related disorders; subarachnoid and aneurismal bleeds, hypotensive shock, concussive inury, septic shock, anaphylactic shock, and sequelae of various encephalitides and meningitides, for example, connective tissue disease-related cerebritides such as lupus. Other uses include prevention of or protection from poisoning by neurotoxins, such as domoic acid shellfish poisoning, neurolathyrism, and Guam disease, amyotrophic lateral sclerosis, Parkinson's disease; postoperative treatment for embolic or ischemic injury; whole brain irradiation; sickle cell crisis; and eclampsia.

A further group of conditions treatable or preventable using tissue protective peptides and peptide analogs of the present invention. include mitochondrial dysfunction, of either a hereditary or acquired nature, which are the cause of a variety of neurological diseases typified by neuronal injury and death. For example, Leigh disease (subacute necrotizing encephalopathy) is characterized by progressive visual loss and encephalopathy, due to neuronal drop out, and myopathy. In these cases, defective mitochondrial metabolism fails to supply enough high energy substrates to fuel the metabolism of excitable cells. A tissue protective peptide or peptide analog optimizes failing function in a variety of mitochondrial diseases. As mentioned above, hypoxic conditions adversely affect excitable tissues. The excitable tissues include, but are not limited to, neuronal tissues such as tissue of the peripheral nervous system (ear and retina) and central nervous system (brain and spinal cord); cardiovascular tissue such as the cells of the heart and associated nerves; and glandular tissue such as the pancreas where T-type calcium channels along with cell-to-cell gap junctions participate in secretion of insulin. An exemplary list of excitable tissue includes, but is not limited to, organs and tissues that include nerves, skeletal muscle, smooth muscle, cardiac muscle, uterus, central nervous system, spinal cord., brain, retina, olfactory system, and auditory system. In addition to the conditions described above, the tissue protective peptides and peptide analogs of the present invention are useful in the treatment of inhalation poisoning such as carbon monoxide and smoke inhalation, severe asthma, adult respiratory distress syndrome, and choking and near drowning. Further conditions which. create hypoxic conditions or by other means induce responsive tissue, such as excitable tissue damage include hypoglycemia that may occur in inappropriate dosing of insulin, or with insulin-producing neoplasms (insulinoma).

Various neuropsychologic disorders which are described to originate from excitable tissue damage are treatable using tissue protective peptides and peptide analogs of the present invention. Chronic disorders in which neuronal damage is involved and for which treatment or preventable by the present invention include disorders relating to the central nervous system andfor peripheral nervous system including age-related loss of cognitive function and senile dementia, chronic seizure disorders, Alzheimer's disease, Parkinson's disease, dementia, memory loss, amyotrophic lateral sclerosis, multiple sclerosis, tuberous sclerosis, Wilson's disease, cerebral and progressive supranuclear palsy, Guam disease, Lewy body dementia, prion diseases, such as spongiform enceptialopathies, e.g., Crcutzfeldt-Jakob disease, Huntington's disease, myoionie dystrophy, Freidrich's ataxia and other ataxias, well as Gilles de La Tourette's syndrome, seizure disorders such as epilepsy and chronic seizure disorder, stroke, brain or spinal cord trauma, AIDS dementia, alcoholism, autism, retinal ischemia, glaucoma, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as unipolar and bipolar affective disorders. Additional neuropsychiatric and neurodegenerative disorders include, for example, those listed in the American Psychiatric Association's Diagnostic and Statistical Manual of Mental Disorders (DSM).

A further group of conditions treatable or preventable using tissue protective peptides and peptide analogs of the present invention include kidney diseases such as renal failure, acute and chronic. Blood supply to the kidneys can be cut off due to several causes including shock from infections invading the bloodstream (septicemia), internal or external hemorrhaging, loss of fluid from the body as a result of severe diarrhea or burns, reactions to transfusions, cardiac arrest or arythmias, surgical trauma and kidney transplantations. The reduced flow of blood to the kidneys resulting from the above conditions may reduced blood flow to dangerously low levels for a time period great enough to cause the development of acute renal failure. The depressed blood flow also results in necrosis, or tissue death, in the kidney, damaging the renal tubular cells. Renal failure may also result from diseases (interstitial and diabetic) nephrotic syndromes, infections, injury (CPB-induced), toxins (contrast-induced, chemotherapy-induced, cyclosporine), autoimmune inflammation (e.g. Lupus, erythrocytosis, etc.) The tissue protective peptides and peptide analogs of the current invention assist in the repair or prevention of this damage helping to ameliorate acute renal failure. Further, the peptides of the current invention may be used to treat, prevent or ameliorate diseases or disorders of the urinary tract including, but not limited, urinary tract infections, irritable bladder, and trauma or radiation injury to the bladder.

The following table lists additional exemplary, non-limiting indications as to the various conditions and diseases amenable to treatment by the aforementioned tissue protective peptides and peptide analogs.

TABLE I
DISEASES AND DISORDERS AMENABLE TO TREATMENT
BY TISSUE PROTECTIVE PEPTIDES AND PEPTIDE ANALOGS

| Cell, tissue, or | Dysfunction or | | |
|---|---|---|---|
| organ | pathology | Condition or disease | Type |
| Heart | Ischemia | Coronary artery disease | Acute, chronic |
| | | | Stable, unstable |
| | | Myocardial infarction | Dressler's syndrome |
| | | Angina | |
| | | Congenital heart | Valvular |
| | | disease | Cardiomyopathy |
| | | Prinzmetal angina | |
| | | Cardiac rupture | Aneurysmatic |
| | | Angiitis | |
| | Arrhythmia | Tachy-, | Stable, unstable |
| | | bradyarrhythmia | Hypersensitive |
| | | Supraventricular, | carotid sinus node |
| | | ventricular Conduction | |
| | | abnormalities | |
| | Congestive heart failure | Left, right, bi- | Cardiomyopathies, |
| | | ventricular, systolic, | such as idiopathic |
| | | diastolic | familial, infective, |
| | | | metabolic, storage |
| | | | disease, deficiencies, |
| | | | connective tissue |
| | | | disorder, infiltration |
| | | | and granulomas, |
| | | | neurovascular |
| | | Myocarditis and | Autoimmune, |
| | | pericarditis | infective, idiopathic |
| | | Cor pulmonale | |
| | Radiation imjury | | |
| | Blunt and penetrating | Intrathoracal adhesions | |
| | trauma | to surgery, infections, | |
| | | or inflammation | |
| | Toxins | Cocaine toxicity, | |
| | | adriamycin, heavy | |
| | | metals (cobalt) | |
| Vascular | Hypertension | Primary, secondary | |
| | Decompression sickness | | |
| | Fibromuscular | | |
| | hyperplasia | | |
| | Aneurysm | Dissecting, ruptured, | |
| | | enlarging | |
| | Cancer | Hemangioma | Hemangiosarcoma, |
| | | hemangiopericytoma | angiosarcoma |
| Lungs | Obstructive | Asthma | |
| | | Chronic bronchitis, | |
| | | Emphysema and | |
| | | airway obstruction | |
| | Ischemic lung disease | Pulmonary embolism, | |
| | | Pulmonary thrombosis, | |
| | | Fat embolism | |
| | Environmental lung | | |
| | diseases | | |
| | Interstitial lung disease | Idiopathic pulmonary | |
| | | fibrosis | |
| | Congenital | Cystic fibrosis | |
| | Cor pulmonale | | |
| | Trauma | | |
| | Pneumonia and | Infectious (including | |
| | pneumonitides | Avian Flu), parasitic, | |
| | | toxic, traumatic, burn, | |
| | | aspiration | |
| | Sarcoidosis | | |
| | Cancers and precancers | | Bronchial carcinooid, |
| | | | oat cell carcinoma |
| | Radiation injury | | |
| Pancreas | Endocrine | Diabetes mellitus, type | Beta cell failure, |
| | | I and II | dysfunction |
| | | | Diabetic neuropathy |
| | | Other endocrine cell | |
| | | failure of the pancreas | |
| | Exocrine | Exocrine pancreas | Pancreatitis |
| | | failure | |
| | Cancer and precancers | Islet cell adenoma, | Islet Cell Carcinoma |
| | | Insulinoma, gastrinoma | |
| Bone | Osteopenia | Primary | Hypogonadism |
| | | Secondary | Immobilization |
| | | | Postmenopausal |
| | | | Age-related |
| | | | Hyperparathyroidism |
| | | | Hyperthyroidism |
| | | | Calcium, magnesium, |
| | | | phosphorus, and/or |
| | | | vitamin D deficiency |
| | Osteomyelitis | | |
| | Avascular necrosis | | |
| | Trauma | | |
| | Paget's disease | | |
| | Cancer | Osteoma | Osteosarcoma |
| Skin | Alopecia | Areata | Primary |
| | | Totalis | Secondary |
| | | | Male pattern baldness |
| | Vitiligo | Localized | Primary |
| | | Generalized | Secondary |
| | Ulceration | Diabetic | Pressure sores, |
| | | Decubitus | pressure ulcers, bed |
| | | Ischemia | sores |
| | Peripheral vascular | Infection, self | |
| | disease | amputation | |
| | Surgical wounds, | | |
| | lacerations | | |
| | Burn injuries | | |
| | Radiation injuries | Cutaneous radiation | |
| | | syndrome | |
| | Cancers and precancers | Nevus, papilloma, | Melanoma, squamous |
| | | seborrheic keratosis, | cell carcinoma, |
| | | skin adnexal tumors | epidermoid |
| | | | carcinoma, basal cell |
| | | | carcinoma and |
| | | | malignant skin |
| | | | adnexal tumors |
| Autoimmune | Lupus erythematosus, | | |
| disorders | Sjogren's syndrome, | | |
| | Rheumatoid arthritis, | | |
| | Glomerulonephritis, | | |
| | Angiitis, Fibromyalgia, | | |
| | Ankylosing spondylitis | | |
| | Langerhans' histiocytosis | | |
| Eye | Optic neuritis | | |
| | Blunt and penetrating | | |
| | injuries, surgical wounds, | | |
| | infections, Sarcoid, | | |
| | Sickle C disease, Retinal | | |
| | detachment, Temporal | | |
| | arteritis | | |
| | Retinal isehemia, | | |
| | Macular degeneration, | | |
| | Retinitis pigmentosa, | | |
| | Arteriosclerotic | | |
| | retinopathy, | | |
| | Hypertensive | | |
| | retinopathy, Retinal | | |
| | artery blockage, Retinal | | |
| | vein blockage, | | |
| | Hypotension, Diabetic | | |
| | retinopathy, glaucoma | | |
| | and Macular edema | | |
| Embryonic and | Asphyxia | | |
| fetal disorders | Ischemia | | |
| | Cancers and precancers | Myxoma, hydatidiform | Myxosarcoma, |
| | | mole | chordoma, |
| | | | choriocarcinoma |
| CNS | Chronic fatigue | | |
| | syndrome, acute and | | |
| | chronic hypo-osmolar | | |
| | and hyperosmolar | | |
| | syndromes, AIDS | | |
| | Dementia, Electrocution | | |
| | Cerebral malaria | | |
| | Encephalitis | Rabies, Herpes | |
| | Meningitis | | |
| | Subdural hematoma | | |
| | Nicotine addiction | | |
| | Drug abuse and | Cocaine, heroin, crack, | |
| | withdrawal | marijuana, LSD, PCP, | |
| | | poly-drug abuse, | |
| | | ecstasy, opioids, | |
| | | sedative hypnotics, | |
| | | amphetamines, | |
| | | caffeine, alcohol | |
| | Obsessive-compulsive | | |
| | disorders | | |
| | Psychotic and depressive | | |
| | disorders | | |
| | Attention deficit and | | |
| | hyperactivity disorders | | |
| | Spinal stenosis, | | |
| | Transverse myelitis, | | |
| | Guillian Barré, | | |
| | Traumatic injury to | | |
| | peripheral nerves, spinal | | |
| | cord, or brain, Nerve root | | |
| | compression, | | |
| | Compression by tumor or | | |
| | vascular malformations, | | |
| | Heat stroke | | |
| | Cancers and precancers | Ganglioneuroma, | Glioma (grades I-III, |
| | | meningioma, | anaplastic, |
| | | schwannoma, | glioblastoma |
| | | neurilemmoma | multiforme (Grade |
| | | | IV), neuroblastoma, |
| | | | medulloblastoma, |
| | | | malignant |
| | | | meningioma, |
| | | | malignant |
| | | | schwannoma |
| ENT | Tinnitus | | |
| | Meunière's syndrome | | |
| | Hearing loss | | |
| | Traumatic injury, | | |
| | barotraumas | | |
| Kidney | Renal failure | Acute, chronic | Vascular/ischemic, |
| | | | interstitial disease, |
| | | | diabetic kidney |
| | | | disease, nephritic |
| | | | syndromes, |
| | | | infections, injury, |
| | | | contrast-induced, |
| | | | chemotherapy- |
| | | | induced, |
| | | | cyclosporine, |
| | | | radiation-induced |
| | | | Cardio Pulmonary |
| | | | Bypass-induced |
| | Radiation injury | | |
| | Henoch | | |
| | Schönlein purpura | | |
| | Cancers or precancers | Renal Tubular | Renal Cell |
| | | adenoma | Carcinoma, |
| | | | hypernephroma |
| Striated muscle | Autoimmune disorders | Myasthenia gravis | |
| | | Dermatomyositis | |
| | | Polymyositis | |
| | Myopathies | Inherited metabolic, | |
| | | endocrine and toxic | |
| | Heat stroke | | |
| | Crush injury | | |
| | Rhabdomyolysis | | |
| | Mitochondrial disease | | |
| | Infection | Necrotizing fasciitis | |
| | Cancers or precancers | Rhabdomyoma | Rhabdomyosarcoma |
| Sexual | Central and peripheral | Impotence secondary | |
| dysfunction | (e.g., erectile dysfunction) | to medication, | |
| | | (diabetes) | |
| Liver | Hepatitis | Viral, bacterial, | |
| | | parasitic | |
| | Ischemic disease | | |
| | Cirrhosis, fatty liver | | |
| | Infiltrative/metabolic | | |
| | diseases | | |
| | Cancers or precancers | Hepatic adenoma | Hepatoma: |
| | | | Hepatocellular |
| | | | carcinoma |
| Gastrointestinal | Ischemic bowel disease | | |
| | Inflammatory bowel | | |
| | disease | | |
| | Necrotizing enterocolitis | | |
| | Wound healing post | abdominal adhesions | |
| | surgical or perforation | due to surgery or | |
| | | infections | |
| | Cancers or precancers | Carcinoid | Maliginant Carcinoid |
| Organ | Treatment of donor, | Transplant rejection, | |
| transplantation | organ and recipient | graft rejection, delayed | |
| | | graft function, graft v. | |
| | | host disease | |
| | Growth of cell or tissue, | | |
| | cultures for tissue | | |
| | regeration, graft or | | |
| | transplantation | | |
| Reproductive | Infertility | Vascular | |
| tract | | Autoimmune | |
| | | Uterine abnormalities | |
| | | Implantation disorders | |
| | Cancers or precancers | | Seminoma, |
| | | | dysgerminoma, |
| | | | choriocarcinoma, |
| | | | embryonal carcinoma, |
| | | | endodermal sinus |
| | | | tumor, |
| | | | teratocarcinoma, |
| | | | Seroli-Leydig tumors, |
| | | | arrhenoblastoma, |
| | | | granulosetheca cell |
| | | | tumors, hilar cell |
| | | | tumors, lipid cell |
| | | | tumors |
| Endocrine | Glandular hyper- and | | |
| | hypofunction | | |
| | Cancers or precancers | Basophilic adenoma, | Parathyroid |
| | | Eosinophilic adenoma, | carcinoma, Medullary |
| | | Chromophobe | carcinoma of thyroid, |
| | | adenoma, Parathyroid | Malignant |
| | | adenoma, C cell | Pheochromocytoma |
| | | hyperplasia, | |
| | | Pheocbromocytoma | |
| General | Shock | Septic, hemodynamic | |
| | Cachexia, Cancer | Anorexia, Asthenia, | |
| | Cachexia | Anemia | |
| | Parasitemia | Malaria, | |
| | | trypanosomiasis, | |
| | | Leshmaniasis | |

As mentioned above, these diseases, disorders or conditions are merely illustrative of the range of benefits provided by the tissue protective peptides and peptide analogs of the present invention. Accordingly, this invention generally provides preventative, therapeutic, or prophylactic treatment of the consequences of mechanical trauma or of human diseases. Prevention or therapeutic or prophylactic treatment for diseases, disorders or conditions of the CNS and/or peripheral nervous system are contemplated. Prevention or therapeutic or prophylactic treatment for diseases, disorders or conditions which have a psychiatric component is provided. Prevention or therapeutic or prophylactic treatment for diseases, disorders or conditions including but not limited to those having an ophthalmic, cardiovascular, cardiopulmonary respiratory, kidney, urinary, reproductive, gastrointestinal, endocrine, or metabolic component is provided. The peptides may be useful for the prevention, therapeutic treatment, prophylactic treatment or management of diseases or disorders associated with tissue damages as well as the damages, effects or symptoms thereof in one or more organs or tissues, preferably at least two, including, but not limited to, the brain, spinal cord, connective tissue, skin, gastrointestinal tract, reproductive organs, liver, heart, lung, kidney, urinary tract, pancreas, eyes and prostate.

In certain embodiments, the methods of the current invention may exclude peptides of the current invention for particular indications. For example, peptides in accordance with Structural Motif C, as described in U.S. Publication No. 2011-0263504, published Oct. 27, 2011, to Cerami et al., which is herein incorporated by reference, may be excluded in methods of the current invention in the indications disclosed within WO 2006/119767 and WO 2007/071248 including: post-operative nerve damage; traumatic nerve damage; spinal cord injury, impaired myelination of nerve fibers; postischemic damage; stroke; Parkinson's disease; Alzheimer's disease; Huntington's disease;, aschizophrenia, dementias; multiple sclerosis, multiinfarct dementias; nerve degeneration associated with diabetes mellitus; neuro-muscular degeneration, disorders affecting the circadian clock or neuro-muscular connections; organ transplantation; genetic or traumatic atrophic muscle disorders; degenerative conditions of the gonads, pancreas, kidney, heart, liver and bowel; diabetes mellitus type I or II; nephrosis; psychoses; neurotic disorders; personality disorders; sexual deviations and disorders; mental retardation; disease in the nervesystem and sense organs; cognitive anomalies; inflammatory disease of the central nervous system; cerebral detzenerations; stimulation of short or long term memory; extra pyramidal diseases and abnormal movement disorders; motor neuron diseases; diseases of the spinal cord; disorders of the autonomic nervous system, diseases of the peripheral nervous system; neuropathies; disorders affecting multiple structures of the eyes; diseases of the ear and mastoid process; abnormalities of organs and soft tissue in newborns; complications of administration of anesthesia or other sedation in labor and delivery; diseases and injuries of the skin; injury to nerves and spinal cord; poisonina by drugs; medicinal and biological substances; metabolic disorders; disorders of endocrine glands; disorders of purine and pyrimidine metabolism; hone disorders; neoplasms; cancers; viral infections of the brain; Gillian-Barre syndromes; pain syndrome; autism and stimulation of the ability to learn. Also, for example, peptides in accordance with Structural Motif D, as described in Publication No. 2011-0263504, published Oct. 27, 2011, to Cerami et al., which is herein incorporated by reference, may be excluded methods of the current invention. in the indications disclosed in U.S. Pat. Nos. 5,571,787, 5,700,909, 5,696,080, 5,714,459, 6,590,074, 6559,124, 6,271,196, 6,268,347, and 6,849,602 including: neuropathic pain due to neuroma (amputation, nerve transaction), nerve compression (entrapment neuropathies, or tumor compression), nerve trauma (crush, stretch, or incomplete transsection); diabetes mellitus; irradiation, ischemia, vasculitis, post-polio syndrome, alcohol, amyloid, toxins, HIV, hypothyroidism, uremia, vitamin deficiencies, chemotherapy, ddC (Zalcitabine), Fabry's diseases, compression (disk, tumor, scar tissue), root avulsion, inflammation (postherpetic neuralgia), spinal cord contusions, spinal cord tumors, spinal cord hemisection, and infarction, tumors or trauma of the brainstem, thalamus or cortex; and demylenathut diseases including multiple sclerosis, acute disseminated leukoencephalitis, progressive multifocal leukoencephalitis, metachromatic leukodystrophy, and adrenal leukodystrophy. For another example, peptides in accordance with Structural Motif E, as described Publication No. 201.1-0263504, published Oct. 27, 2011, to Cerami et al., which is herein incorporated by reference, may be excluded in methods of the current invention in the indications disclosed in U.S. Pat. No. 7,259,146 and. US Patent Publication No. 20030130197, including: acute neurodegenerative disorders: cerebral ischemic or infarction including embolic occlusion and thrombotic occlusion; reperfusion following acute ischemia; perinatal hypoxic-ischemic injury; cardiac arrest; intracranial hemorrhage; intracranial and intravertebral lesions; and whiplash shake infant syndrome; chronic neurodegenerative disorders; Alzheimer's disease, Pick's disease, diffuse Lewy body disease, progressive suprenuclear palsy, multisystem degeneration, chronic epileptic conditions, motor neuron diseases, prion diseases, neurological and psychiatric manifestations associated with peripheral diseases including EPO deficiency, blood loss, renal failure, endstage renal disease, renal transplant,and other diseases associated with anemia including hematological and non-hematological malignancies/tumors, complications associated with chemotherapy and other drugs, hematological disorders, inflammatory and infectious disorders, chronic systemic autoimmune diseases, Hencoh Schonlein Purpura hemolytic uremic syndrome, chemical, toxic, infectious, and radiation injury of the nervous system, and encephalopathies; plexopathies; neuropathies; Charcot-Marie-Tooth disease; Friedreich's ataxia; metachromatic leukodystrophy; Refsum's disease; adrenomycloneuropathy; Ataxia-telangiectasia; Djerine-Sottas neuropathy; Lambert-Eaton syndrome; and disorders of the cranial nerves. As a further example, peptides in accordance with Structural Motif F, as described in U.S. Publication No. 2011-0263504, published Oct. 27, 2011, to Cerami et al., which is herein incorporated by reference, may be excluded in methods of the current invention in the indications disclosed in WO/2007/052154 including: immune-mediated inflammation; autoimmune diseases including Hashimoto's thyroiditis, insulin dependent diabetes mciiitus, systemic lupus erythmatosus; demylenating disease including multiple sclerosis, traverse myelitis. Guillain-Barre syndrome, and progressive multifocal leukoenccphalopathy and demylenation resulting from organophosphate exposure; arthritis; acute cerebrovascular injury; acute spinal cord injury; acute brain injury; acute cardiovascular injury; stroke; traumatic injury; transplant rejection; and graft rejection.

C. Prevention, Treatment, Amelioration, or Management of the Damage, Effects, or Symptoms of Diseases, Disorders or Conditions.

In a further embodiment of the invention, the method of treatment of the current invention is useful for preventing, treating, ameliorating, or managing the damage, effects, or symptoms of the above noted diseases and disorders. In particular, the current method of treatment can be used to address symptoms including, but not limited to, cachexia, carcinogenesis, sterilization, cataract formation, radiodermatitis, beta burns, gamma burns, loss of cells (in particular bone marrow, digestive tract cells), damage to the hematopoietic, gastrointestinal, central nervous, cardiovascular, skin, and/or reproductive systems, acute radiation syndrome (feeling of nausea, vomiting, general illness and fatigue, immune system depression, loss of hair, uncontrollable bleeding (mouth, under the skin, kidneys), massive diarrhea, delirium, coma and death), chronic radiation syndrome, cutaneous radiation syndrome (inflammation, erythema, dry or moist desquamation, hair loss, blistering, reddening, ulceration, damage to sebaceous and sweat glands, atrophy, fibrosis, decreased or increased skin pigmentation, and necrosis), headaches, dizziness, nausea, vomiting, mucosal irritation, dysponea, impaired consciousness, coma, convulsions, tachy- and brady-dysrhythmias, hypotension, cardiovascular collapse, acyanosis. bradycardia, myosls, excessive salivation, diarrhea, involuntary micturition, muscle fasciculation, initial depolarizing flaccid paralysis, spike discharges and convulsions, intermediate syndrome, neurotoxic esterase inhibition, organophosphate-induced delayed neuropathy, erythema, edema, necrosis and vesicles, melanoderma tracheobronchitis, bronchospasms, bronchial obstruction, hemorrhagic pulmonary edema, respiratory failure, bacterial pneumonia, eye erythema, lachrymation, discomfort of the eyes, severe pain in the eyes, blepharospasm, iritis, blindness, bone marrow suppression, lewisite shock, hepatic necrosis, renal failure secondary to hypoperfusion, burning sensations (eyes, nasopharynx, oropharynx), profuse tearing, rhinorrhoea, coughing hoarseness, dyspnoea, odynophagia conjunctivitis, conical injury, naso-orophangyal injury/edema, respiratory distress due to inflammation of the glottic structures, secretions, and/or lyrangospasms, acute respiratory syndromes, disorientation, behavioral modifications, and reactive airway dysliniction syndrome.

As mentioned above, these diseases or disorders associated with tissue damage or damage, effects, or symptoms resulting therefrom, are merely illustrative of the range of disorders that can be addressed by the peptides used in the method. of the current invention. Accordingly, this invention generally provides preventative, therapeutic, or prophylactic treatment of a disease or disorder associated with tissue damage or damage, effects or symptoms resulting therefrom.

Diseases or disorders associated with tissue damage or damage, effects or symptoms resulting therefrom. can be treated or prevented by administration of an effective amount of a peptide of the invention. In certain embodiments, the present invention provides methods of treating or preventing a disease or disorder described herein. comprising the step of administering to a subject having the disease or disorder an amount of a peptide of the invention effective to treat or prevent the disease or disorder. In one ethbodiment, a composition comprising an effective amount of one or more peptides of the invention, or a pharmaceutically acceptable salt thereof, is administered.

D. Treatment in Conjunction with Other Therapeutics for a Cumulative or Synergistic Effect.

In certain embodiments, the invention encompasses methods for treating, mediating, ameliorating or preventing a disease or disorder associated with tissue damage or damage, effects or symptoms resulting therefrom, comprising administering to a patient in need thereof an effective amount of a peptide and another suitable therapeutic agent, each being administered according to a regime suitable for the medicament. This may be done to achieve additive, synergistic or offsetting (to counteract side effects of the therapeutic) benefits of the effects of the peptide and therapeutic agents. This includes the concurrent, substantially simultaneous, or non-concurrent administration of the peptide and suitable therapeutic agent. The non-concurrent administration of the peptide and a suitable therapeutic agent includes sequential, alternating, and acute vs. chronic administration of the peptides and suitable therapeutic agents. Also, the peptide and the suitable therapeutic agent may be administered in the same or separate pharmaceutical compositions, and if administered separately they may be administered via the same route of administration or different routes. Suitable therapeutic methods and agents may include, but are not limited to, carbamates (pyridostigmine, physostigmine, aminostigmine, neostigmine, synostigmine, Epastigmine, Mobam, decarbofuran), anticholingeries (trihexyphenidyle, benactyzine, Biperidene, Scopolamine, aprophen, atropine, hyoscin, adiphenine, Caramiphen, pentmethonium, Mecamylamine, Tribexyphenidyle) PANPAL, aminophenols (eseroline), organophosphates (TEPP, Paraxon, Ethyl-4-nitrophenylphosphate), tacrine, 7-MEO-TA, huperzine A, Cholinesterases (BuChE, AChE, triesterase, paraoxonase), oximes/reactivators (HI-6, PAM, Obidoxime, Trimedoxime, Methoxime, Hlo-7, BI-6, K048, K033, pralidoxime chloride (2-PAM CI), P2S, TMB4, 2-PAMI), Suramine, Benzodiazepines, tubocurine, Memantine, Procyclidine, Nimodipin, Clonidine, pralidoxime, diazepam, enkephalins, phonylmethylsulfonyl fluoride, natrium bicarbonate, vitamin E analogs (α-tocopherol succinate, γ-tocotrienol), superoxide dismutase/catalase mimic (EUK189), selenium, benzyl styryl, sulfone, truncated flagellin, statins, genistein, galantamine. hypothermia, 5-androstenediol, CpG-oligodeoxynucleotides, antimicrobials, stem cell transplants, amifostine, Tempol, isoflavones, benzylsullone analogs, GM-CSF, G-CSF, potassium iodide, aluminum hydroxide, Prussian blue, chelating agents (diethylenetriaminepentaacetate (Ca-DTPA), zinc diethylenetriaminepentaacetate (Zn-DTPA)), keratinocyte growth factor, intestinal peptide hormones, beta glucan, octreotide, pentoxifylline, angiotensin converting enzyme inhibitors, angiotensin II receptor blockers, methemoglobin formers (amyl nitrite, sodium nitrite), sodium thiosulfate, cobalt compounds (hydroxycobalamin (Vitamin B12a), toxoids, antitoxins, vaccines, passive antibodies, chemotherapeutic agents including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, daunorubicin, dactinomycin, mitoxantrone, asparaginase, vinblastine, vincristine, vinorelbine, paclitaxel, and docetaxel; Radiation: γ-radiation; Alkylating agents; Nitrogen mustards: cyclophosphamide, Ifosfamide trofosfamide, Chlorambucil; Nitrosoureas: carmustine (BCNU), Lomustine (CCNU), Alkylsulphonates busulfan, Treosulfan; Triazenes: Dacarbazine; Platinum containing compounds: Cisplatin carboplatin, Plant Alkaloids; Vinca alkaloids: vincristine, Vinblastine, Vindesine, Vinorelbine; Taxoids: paclitaxel, Docctaxol; DNA Topoisomerase Inhibitors Epipodophyllins: etoposide, Teniposide, Topotecan, 9-aminocamptothecin irinotc;can (Campto crisnatol; Mytomycins: Mytomycin C, Mytomycin C; Anti-metabolites, Anti-folates: DHFR inhibitors: methotrexate, Trimetrexate; IMP dehydrogenase Inhibitors: mycophenolic acid, Tiazofurin, Ribavirin EICAR; Ribonuclotide reductase Inhibitors: hydroxyurea; deferoxamine; Pyrimidine analogs: Uracil analogs, 5-Fluomuracil, Doxifluridine, Ratitrexed; Cytosine analogs: cytarabine (ara C) Cytosine arabinoside fludarabine; Purine analogs: mercaptopurine, Thioguanine; Hormonal therapies; Receptor antagonists: Anti-estrogens, Tamoxifen, Raloxifene megestrol; LHRH agonists: goserelin, Leuprolide acetate; Anti-androgens: flutamide, bicalutamide; Retinoids/Deltoids Vitamin D3 analogs: EB 1089, CB 1093, KB 1060; Photodyamic therapies: vertoporfin (BPD-MA), Phthalocyanine photosensitizer, Pc4 Demethoxy-Itypocrellin A (2BA-2-DMHA) Cytokines: Interferon-α, interferon-γ, Tumor necrosis factor; Isoprenylation inhibitors: Lovastatin; Dopaminergic neurotoxins: 1-methyl-4-phenylpytidinium ion; Cell cycle inhibitors: staurosporine; Actinomycins: Actinomycin D, Dactinomycin; Bicomycins: bleomycin A2, Bleomycin B2, Peplornycin; Anthracyclines: datmorubicin, Doxorubiein (adriamycin), Idarubicin, Epirubicin, Pirarubicin, Zorubicin, Mitoxantronc; MDR inhibitors: verapamil; Ca.sup2+ ATPase inhibitors: thapsigargin; INF-α inhibitors/thalidomide angiogenesis inhibitors 3-(3,4-dimethoxy-phenyl)-3-(1-oxo-1,3-dihydro-isoindol-2-yl)- propionamide (SeICIDs™) ImiDs™, Revlimid™, Actimid™. In another aspect of the present invention, a pharmaceutical composition according to the present invention may include a peptide in a formulation with at least one small molecule that exhibits tissue protective functionality. Suitable small molecules include, but are not limited to, steroids (e.g., lazaroids and glucocorticoids), antioxidants (e.g., coenzyme Q₁₀, alpha lipoid acid, and NADH), anticatabolic enzymes (e.g., glutathione peroxidase, superoxide ditnutase, catalase, synthetic catalytic scavengers, as well as mimetics), indole derivatives (e.g., indoleamines, carbazoles, and carbolines), nitric acid neutralizing agents, adenosine adenosine agonists, phytochemicals (flavanoids), herbal extracts (ginko biloba and turmeric), vitamins (vitamins A, E, and C), oxidase electron acceptor inhibitors (e.g., xanthine oxidase electron inhibitors), minerals (e.g., copper, zinc, and magnesium), non-steriodal anti-inflammatory drugs (e.g., aspirin, naproxen, and ibuprofen), and combinations thereof. Additionally agents including, but not limited to, anti-inflammatory agents (e.g., corticosteroids, prednisone and hydrocortisone), glucocorticoids, steroids, non-stcriodal anti-inflammatory drugs (e.g., aspirin, ibuprofen, diclofenac, and. COX-2 inhibitors), beta-agonists, antieholinergie agents and methyl xanthines), immunomodulatory agents (e.g., small organic molecules, T cell receptor modulators, cytokine receptor modulators, T-cell depicting agents, cytokine antagonists, monokine antagonists, lymphocyte inhibitors, or anti-cancer agents), gold injections, sulphasalazine, poniedlamine, anti-angiogenic agents (e.g., angiostatin), TNF-α antagonists (e.g., anti-TNFα antibodies), and endostatin), dapsone, psoralens methoxalen and trioxsalen), anti-malarial agents (e.g., hydroxychloroquine), anti-viral agents, anti-histamines and antibiotics (e.g., erythromycin and penicillin) may be used in conjunction with the current pharmaceutical compositions.

In other embodiments, the present methods for treating, mediating, ameliorating or preventing a disease or disorder associated with tissue damage or damage, effects or symptoms resulting therefrom further comprise administration of the peptides in conjunction with methods of treatment such as chemotherapy, radiation therapy (x-ray radiation, high-energy megavoltage (radiation of greater that 1 MeV energy), electron beam, orthovoltage x-ray radiation, gamma-ray emitting radioisotopes (radioactive isotopes of radium, cobalt and other elements)), hyperbaric chambers, heart bypass machine, angioplasty, hypothermia, surgery, angioplasty, etc. to to achieve additive, synergistic or offsetting (to counteract side effects of the therapeutic method) benefits of the effects of the peptide and therapeutic method. As an example, in a specific embodiment, peptide can be administered to a patient that has undergone surgery as treatment for the cancer concurrently with chemotherapy or radiation therapy. In another specific embodiment, a chemotherapeutic agent or radiation therapy is administered prior or subsequent to administration of a peptide, preferably at least an hour, five hours, 12 hours, a day, a week, a month, more preferably several months (e.g., up to three months). Additionally, the invention provides methods of treatment of cancer or neoplastic disease with a peptide as an alternative to chemotherapy or radiation therapy where the chemotherapy or the radiation therapy has proven or may prove too toxic, e.g., results in unacceptable or unbearable side effects, for the patient being treated. Alternatively, the invention provides methods of treatment wherein the peptide is administered prior to, simultaneously with or following treatment with chemotherapy or radiation in an effort to prevent or ameliorate the toxic side effects of the treatment method. As demonstrated in Example 2, the peptides administered in accordance with the current method are able to ameliorate the side-effects of cis-platinum a known chemotherapeutic. Although, the above examples relate to the treatment of cancers, it is understood that the peptides may be administered in conjunction with other methods of treatment in the art for diseases or disorders associated with tissue damage and damage, effects, or symptoms resulting therefrom including inflammation, and exposure to toxic agents to achieve synergistic, additive or offsetting results.

E. Formulation and Administration of Peptides

In one embodiment, the method of the current invention provides that a pharmaceutical composition comprising a peptide can be administered systemically to protect or treat the targeted cells, tissue or organ. Such administration may be parenterally, via inhalation, or transmucosally, e.g., orally, bueally, nasally, rectally, intravaginally, sublingually, ocularly, submucosally or transdennally. Preferably, administration is parenteral, e.g., via intravenous or intraperitoneal injection, and also including, but is not limited to, intra-arterial, intramuscular, intradermal and subcutaneous administration.

For other routes of administration, such as by use of a perfusate, injection into an organ, or other local administration, a pharmaceutical composition will be provided which results in similar levels of a peptide as described above. A level of about 15 pM-30 nM is preferred.

The pharmaceutical compositions of the invention may comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica. gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release fun and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triohylamine 2-ethylamino ethanol, histidine, procaine, etc. Examples of suitable phannaceittical carriers are described in “Remington's Pharmaceutical Sciences” by E. W. Martin, hereby incorporated by reference herein in its entirety. Such compositions will contain a therapeutically effective amount of the compound, preferably in purified form, together with a suitable amount of carrier so as to provide the form for proper administration to the patient. The formulation should suit the mode of administration.

Formulations for increasing transmueosal adsorption of peptides such as long acting peptides are also contemplated. by the current invention. Pharmaceutical compositions adapted. for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch, or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral. administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops, In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation of compounds directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets. in one embodiment, an autoinjector comprising an injectable solution of a peptide may be provided for emergency use by ambulances, emergency rooms, and battlefield situations, and even for self-administration in, a domestic setting, particularly where the possibility of traumatic amputation may occur, such as by imprudent use of a lawn mower. The likelihood that cells and tissues in a severed foot or toe will survive after reattachment may be increased by administering a peptide to multiple sites in the severed part as soon as practicable, even before the arrival of medical personnel on site, or arrival of the afflicted individual with severed toe in tow at the emergency room.

In a preferred embodiment, the composition is formulated in accordance with S routine procedures as a pharmaceutical .composition adapted for intravenous administration to human beings. Typically, compositions for intravenous administration are solutions in sterile isotonic aqueous buffer. Where necessary the composition may also include a solubilizing agent and a local anesthetic such as lidocaine to ease pain at the site of the injection. Generally, the ingredients are supplied either separately or mixed together in unit dosage form, for example. as a dry lyophilized powder or water-free concentrate in a hermetically-sealed container such as an ampule or sachette, indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A pertitsate composition may be provided for use in situ perfusion. Such pharmaceutical compositions may comprise levels of peptides, or a form of peptides not suitable for acute or chronic, local or systemic administration to an individual, but will serve the functions intended herein in as an organ bath, organ perfusate, or in situ perfusate prior to removing or reducing the levels of the peptide contained therein before exposing or returning the treated organ or tissue to regular circulation.

The invention also provides a pharmaceutical pack or kit con one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, for example, a peptide can be delivered in a controlled-release system. For example, the peptide may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507: Saudek et al., 1989, N. Engl. J. Med. 321:574, each of which is incorporated by reference herein in its entirety). In another embodiment, the compound can be delivered in a vesicle, in particular a liposome (see Langer, Science 249:1527-1533 (1990); Treat et al., in Liposomes in the Therapy of infectious Disease and Cancer, Lopez-Berestein and Fidler (eds.), Liss, New York, pp. 353-365 (1989); WO 91/04014; U.S. Pat. No. 4,704355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974: Controlled Drug Bioavailability, Drug Product Design and Performance, Smolen and Ball (eds.), Wiley: New York (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard et al., 1989, J. Neurosurg. 71:105, (each of which is incorporated by reference herein in its entirey).

In yet another embodiment, a controlled release system can be placed in proximity of the therapeutic target, i.e., the target cells, tissue or organ, thus requiring only a Fraction of the systemic dose (see, e.g., Goodson, pp. 115-138 in Medical Applications of Controlled Release, vol. 2, supra, 1984, which is incorporated by reference herein in its entirety). Other controlled release system are discussed in the review by Langer (1990, Science 249:1527-1533, which is incorporated by reference herein in its entirety).

In another embodiment, peptide, as properly formulated, can be administered by nasal, bucal, oral, rectal, vaginal, ocular, transdermal, parenteral, inhalation or sublingual administration.

In a specific embodiment, it may be desirable to administer a peptide of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. A non-limiting example of such an embodiment would be a stent or other scaffolding coated with a peptide of the present invention implanted in a portion of the vasculature, duct, etc.

Selection of the preferred effective dose will be readily determinable by a skilled artisan based upon considering several factors, which will be known to one of ordinary skill in the art. Such factors include the particular form of peptide, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

In another aspect of the invention, a perfusate or perfusion solution is provided for perfusion and storage of organs for transplant, the perfusion solution includes an amount of a peptide or peptide analog effective to protect responsive cells and associated cells, tissues or organs. Transplant includes but is not limited to allotransplantation, where an organ (including cells, tissue or other bodily part) is harvested from one donor and transplanted into a different recipient, both being of the same species; autotransplantation, where the organ is taken from one part of a body and replaced at another, including bench surgical procedures, in which an organ may be removed, and while ex vivo, resected, repaired, or otherwise manipulated, such as for tumor removal, and then returned to the original location or xenotransplantation, where tissues or organs or transplanted between species. In one embodiment, the perfusion solution is the University of Wisconsin (UW) solution (U.S. Pat. No. 4,798,824, hereby incorporated by reference herein in its entirety) which contains 5% hydroxyethyl starch (having a molecular weight of from about 200,000 to about 300,000 and substantially free of ethylene glycol, ethylene chlorohydrin, sodium chloride and acetone); 25 mM KH₂PO₄: 3 mM glutathione; 5 mM adenosine; 10 mM glucose; 10 mM HEPES buffer; 5 mM magnesium gluconate; 1.5 mM CaCl₂, 105 mM sodium gluconate; 200,000 units penicillin; 40 units insulin; 16 mg dexamethasone; 12 mg Phenol Red; and has a pH of 7.4-7.5 and an osmolality of about 320 mOsm/l supplemented with an appropriate amount of a peptide of the invention. This particular perfusate is merely illustrative of a number of such solutions that can be adapted for the present use by inclusion of an effective amount of a peptide. In a further embodiment, the perfusate solution contains from about 1 to about 500 ng/ml of a peptide, or from about 40 to about 320 ng/ml peptide. As mentioned above, any form of peptide can be used in this aspect of the invention.

While the preferred recipient of a peptide for the purposes herein throughout is a human, the methods herein apply equally to other mammals, particularly domesticated animals, livestock, companion, and zoo animals. However, the invention is not so limiting and the benefits can be applied to any mammal.

In further aspects of the ex-vivo invention any peptide such as but not limited to the ones described above may be employed.

In another aspect of the invention, methods and compositions for preventing, treating or managing a disease or disorder associated with tissue damage or damage, effects or symptoms resulting therefrom in cells, tissues or organs which are not isolated from the vasculature by an endothelial cell barrier are provided by exposing the cells, tissue or organs directly to a pharmaceutical composition comprising a peptide, or administering or contacting a pharmaceutical composition containing a peptide to the vasculature of the tissue or organ.

Similar to other tissue protective compounds based on erythropoietin, it is possible that the peptides of the present invention may be transported from the luminal surface to the basement membrane surface of endothelial cells of the capillaries of organs with endothelial cell tight junctions, including, for example, the brain, retina, and testis. Thus, the effects of a disease or disorder associated with tissue damage or damage, effects or symptoms resulting therefrom on cells across the barrier may be treated. While not wishing to be bound by any particular theory, after transcytosis of the peptide may interact with a tissue-protective receptor on a cell, for example, neuronal, eye (e.g., retinal), adipose, connective, hair, tooth, mucosal pancreatic, endocrine, aural, epithelial, skin, muscle, heart, lung, liver, kidney, small intestine, adrenal (e.g. adrenal cortex, adrenal medulla), capillary, endothelial, testes, ovary, stem or endometrial cell, and receptor binding can initiate a signal transduction cascade resulting in the activation of a gene expression program within the responsive cell or tissue, resulting in the protection of the cell or tissue, or organ, from damage, such as by exposure to a toxic agent, inflammation, hypoxia, etc. In another embodiment, the peptide can be cross-linked to a compound that can cross the barrier, such as CEPO, to be transported across the barrier in accordance with the teaching of PCT Application No. PCT/US01/49479, U.S. patent application Ser. Nos. 10/188,905 and 10/185,841, incorporated herein by reference.

Thus, methods for protecting a tissue from disease or disorder associated with tissue damage or damage, effects or symptoms resulting therefrom are described in detail herein below.

In the practice of one embodiment of the invention, a mammalian patient is undergoing systemic chemotherapy for cancer treatment, including radiation therapy, which commonly has adverse effects such as nerve, lung, heart, ovarian or testicular damage.

Administration of a pharmaceutical composition comprising a tissue protective peptide or peptide analog as described above is performed prior to and during chemotherapy and/or radiation therapy, to protect various tissues and organs from damage by the chemotherapeutic agent, such as to protect the testes. Treatment may be continued until circulating levels of the chemotherapeutic agent have fallen below a level of potential danger to the mammalian body.

In the practice of another embodiment of the invention, various organs are planned to be harvested from a victim of an automobile accident for transplant into a number of recipients, some of which required transport for an extended distance and period of time. Prior to organ harvesting, the donor is infused with a pharmaceutical composition comprising tissue protective peptides and peptide analogs as described herein. Harvested organs for shipment are perfused with a perfusate containing tissue protective peptides or peptide analogs as described herein, and stored in a bath comprising tissue protective peptides or peptide analogs. Certain organs are continuously perfused with a pulsatile perfusion device, utilizing a perfusate containing tissue protective peptides and peptide analogs in accordance with the present invention. Minimal deterioration of organ function occurs during the transport and upon implant and reperfusion of the organs in situ.

In another embodiment of the present invention, a participant in a hazardous activity that exposes the individual to toxic agents, one could take a dose of a pharmaceutical composition containing a peptide ,sufficient to either prevent (i.e. delaying the onset of, inhibiting, or stopping), protect against, or mitigate the effects of exposure to a toxic agent. In particular, this method of treatment may have application in various professions involving contact with toxic agents, such as miners, chemical manufacturers, military personnel (soldiers, paratroopers), emergency personnel (police, fire, EMS, and disaster relielpersonnel.), construction workers, food processors, and employees at power reactors.

In another embodiment: of the invention, a surgical procedure to repair a heart valve requires temporary cardioplegia and arterial occlusion. Prior to surgery, the patient is infused with a tissue protective peptide or peptide analog. Such treatment prevents hypoxic ischemic cellular damage, particularly after reperfusion. Additionally, the pharmaceutical compositions of the present invention may be used prophylactically to prepare an individual for surgery in an effort to limit the trauma associated with the surgical procedure or aide in the recovery of the individual from the surgical procedure. Although the present method of treatment using pharmaceutical compositions containing tissue protective peptides and peptide analogs provide a pmphylactic use for surgical procedures, it may be particularly useful in procedures that induce temporary ischemic events including, but not limited to, bypass procedures (coronary bypass), angioplasty procedures, amputations, and transplantations, as well as, those performed directly upon responsive cells, tissues, or organs such us brain and spinal cord surgery, and open heart procedures. Such procedures may involve the use of S cardiopulmonary (heart lung) bypass.

In another embodiment of the invention, in any surgical procedure, such as in cardiopulmonary bypass surgery, a tissue protective peptide or peptide analog of the invention can be used. In one embodiment, administration of a pharmaceutical composition comprising tissue protective peptides and peptide analogs as described above is performed prior to, during, and/or following the bypass procedure, to protect the function of brain, heart, and other organs.

In the foregoing examples in which a peptide is used for ex-vireo applications, or for in vivo applications to treat a disease or disorder associated with tissue damage or damages, effects or symptoms resulting therefrom, the invention provides a pharmaceutical composition in dosage unit form adapted for prevention, treatment or management of the damages and effects of exposure to a toxic agent or symptoms thereof which comprises an amount within the range from about 0.01 pg to 30 mg, 0.5 pg to 25 mg, 1 pg to 20 mg, 500 pg to 10 mg, 1 ng to 10 mg, 500 ng to 10 mg, 1 ug to 10 mg, 500 μg to 10 mg, or 1 mg to 10 mg of a peptide, and a pharmaceutically acceptable carrier. In a preferred embodiment, the amount of peptide is within the range from about 0.5 pg to 1 mg. In a preferred embodiment, the formulation contains peptides that are non-erythropoietic.

Furthermore, this restorative aspect of the invention is directed to the use of any peptides herein for the preparation of a pharmaceutical composition for the restoration of cellular, tissue or organ dysfunction, wherein treatment is initiated after, and well after, the initial insult responsible for the dysfunction. Moreover, treatment using peptides of the invention can span the course of the disease or condition during the acute phase as well as a chronic phase.

A peptide of the invention may be administered systemically at a dosage between about 1 ng and about 300 μg /kg body weight, preferably about 5 -150 μg/kg-body weight, most preferably about 10-100 μg/kg-body weight, per administration. For example, administration may be repeated hourly, daily, as long as clinically necessary, or after an appropriate interval, e.g., every 1-12 hours, preferably every 6 to 12 hours; every 2-6 days, preferably every 2-4 days; every 1 to 12 weeks, preferably, every 1 to 3 weeks. In one embodiment, the effective amount of peptide and a pharmaceutically acceptable carrier may be packaged in a single dose vial or other container. In another embodiment, the peptides, which are capable of exerting the activities described herein but not causing an increase in hemoglobin concentration or hematocrit, are used. Such peptides are preferred in instances wherein the methods of the present invention are intended to be provided chronically,

### EXAMPLES

### Example 1

### Method of Peptide Synthesis

RYLLEAKEAENITTG (SEQ ID NO:1) can be synthesized using standard Fmoc solid phase peptide synthesis on Wang resin, purified by preparative HPLC and ion-exchange chromatography, and lyophilized. Acetate and ammonium are bound in, ionic form to basic and acidic groups of the peptide molecule forming a mixed salt.

### Example 2

### Peptide is Active in Sciatic Nerve Injury Assay

RYLLEAKEAENITTG (SECS ID NO:1) was tested for tissue protective activity using a sciatic nerve injury assay, Sprague-Dawley rats (250-300 grams) (six per group, including control) were anesthetized using isoflurane. The rat was then placed on a homeothermic blanket to ensure that the core temperature of the rat was maintained at 35-37° C. during the operation. Core temperature was monitored via a rectal probe. The right sciatic nerve of the anesthetized rat was exposed at mid thigh through a quadriceps muscle dissection; a 2 cm incision with a 15 blade scalpel was made through the skin parallel and over the quadriceps muscle and the quadriceps muscle was cut to expose the sciatic nerve using a pair of dissecting scissors. The sciatic nerve was then freed from the surrounding membranes. A 2-0 braided silk thread (Ethicon, 685-G) was passed under the nerve and the ends of the suture passed through a guide which was maintained perpendicular to the nerve. The end of the suture was then tied to a non-elastic cord which was then draped around the pulley system (a NYL pulley bearing MTD (Number 04174-01) with stabilizer) and a 100 gram weight attached to the non-elastic cord was slowly released. The weight was allowed to hang for 1 minute before the silk suture was cut to release the weight.

A 50 mcg/kg dose of RYLLEAKEAENITTG (SEQ ID NO:1) or PBS was then injected into the caudal vein using a ½ cc insulin syringe. The muscle and surgical incision were S then closed and 5 ml of Lactated Ringers solution was injected subcutaneously into the rat. The core temperature of the rat was maintained at 35-37° C. using a heat blanket during recovery.

Over the next four days the rear toe splaying of the rats was determined by placing the rat in an acrylic tube with a diameter of 30 cm on the scanning surface of a digital scanner. After waiting 5 minutes in order to permit acclimation, a scan was taken of the rat's back feet that clearly displayed all 5 toes. Three acceptable scans of each rat were taken. From the scans, the Toe Spread (the distance between the ball of the first toe and the ball of the fifth toe) and the Intermediate Toe Spread (the distance between the ball of the second toe and the ball of the fourth toe were measured. The static sciatic index (SSI) was then computed in accordance with S. Erbayraktar et al., 2003, Proc. Natl. Acad Sci USA 100, 6741-6746 (hereby incorporated by reference in its entirety) and statistical analysis performed.

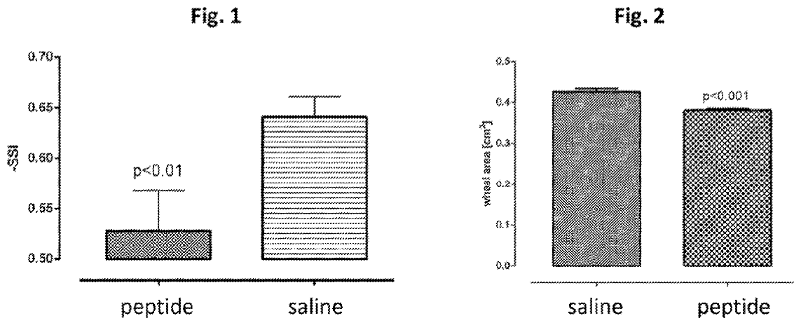

FIG. 1 shows that RYLLEAKEAENITTG (SEQ ID NO:1) reduces sciatic nerve injury in this assay.

### Example 3

### Histamine Induced Wheal Formation

Under isofiurane anesthesia, 12 Sprague-Dawley rats' abdomens were shaved and depilated. Each rat was then injected intravenously (via internal jugular) with a dilute solution or Evans Blue (30 mg/ml in saline, 1 mg/kg bw). After 5 minutes, 6 small doses of histamine (histamine diphosphate, 20 microliters administered intradermally) in a rectangular pattern on each rat's abdomen. After fifteen minutes, when the wheal reaches its maximum size the wheal is photographed and the blister area was determined by digital planimetry. To test the efficacy of a peptide, RYLLEAKEAENITTG (SEQ ID NO:1) or placebo, was administered to the rats intravenously shortly after the histamine injection, at a dose of 30 mcg/kg. As shown in FIG. 2, the wheal area was significantly reduced by RYLLEAKEAENITTG (SEQ ID NO:1).

### Example 4

### Cachexia Model

Male Wistar Han rats aged 8 weeks with an approximate weight of 200 g were inoculated intraperitoneally with 10⁸ Yoshida hepatoma. AH-130 cells. Body weight and body composition were assessed before tumor inoculation and on the day of sacrifice. Quality of life indicators (spontaneous activity, food and water intake) were measured on day 0 and on day 10/11. Tumor cells were counted at the last day of the study. Rats were treated daily with either (1) low dose (0.17 μg/kg/d) RYLLEAKEAENITTG (SEQ ID NO:1), (2) high dose (1.7 μg/kg/d) RYLLEAKEAENITTG (SEQ ID NO:1), or (3) or normal saline (placebo) (n=22). Body composition of animals was analyzed using an EchoMRI-700 (Echo Medical Systems, Houston, Tex., USA). The analysis of the body structures was based on Nuclear Magnetic Resonance. Spontaneous activity was measured by an infrared scanner over 24 hours using Supermex activity monitoring system (Muromachi Kikai Co., LTD., Tokyo, Japan).

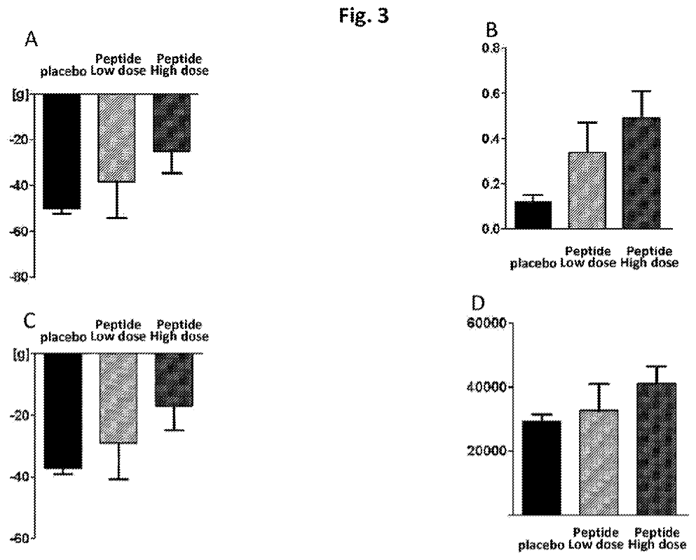

Cancer cachexia is a severe complication supporting the last stages of the disease and characterized by the substantial loss of muscle mass often accompanied by the loss of fat. Treatment with RYLLEAKEAENITTG (SEQ ID NO:1) reduced weight loss (FIG. 3A), preserved epididymal fat (FIG. 3B), reduced loss of lean mass (FIG. 3C) and increased physical activity (FIG. 3D), thus reducing the adverse effects of cachexia.

What is claimed is:

1. An isolated peptide comprising a 15 to 29-residue peptide or peptide analog having tissue protective activity in a responsive cell, tissue, or organ, and an amino acid sequence of RYLLEAKEAENITTG (SEQ ID NO:1), wherein one amino acid residue is optionally replaced with a conservative or nonconservative substitution which is an amino acid or amino acid equivalent having a side chain modification or side chain substitution.

2. The isolated peptide of claim 1, wherein the amino acid sequence of the peptide consists of RYLLEAKEAENITTG (SEQ ID NO:1).

3. A pharmaceutical composition comprising the isolated peptide of any one of claims 1-2 and a pharmaceutically acceptable carrier.

4. The pharmaceutical composition of claim 3, wherein the composition is formulated for oral, intranasal, ocular, inhalational, transdermal, rectal, sublingual, or parenteral administration.

5. The pharmaceutical composition of claim 3, wherein the composition is formulated as a perfusate solution.

6. A method of treating a subject suffering from a disease associated with tissue damage, the method comprising the step of administering to the subject an effective amount of the isolated peptide of any one of claims 1-2 or the pharmaceutical compositions of claim 3.

7. The method of claim 6, wherein the disease associated with tissue damage is caused by cancer, ischemic, traumatic, toxic, or inflammatory injuries.

8. The method of claim 6, wherein the disease associated with tissue damage is a cardiovascular disease, cardiopulmonary disease, respiratory disease, kidney disease, disease of the urinary system, disease of the reproductive system, bone disease, skin disease, gastrointestinal disease, endocrine abnormality, metabolic abnormality, cognitive dysfunction, or a disease or disorder of the central or peripheral nervous system.

9. A method for preventing, treating, ameliorating, or managing cancer, a neoplastic disorder, inflammation, or toxic agent exposure in a subject in need thereof comprising administration to said subject an effective amount of an isolated peptide.' comprising a 15 to 29-residue peptide or peptide analog having tissue protective activity in a responsive cell, tissue, or organ, and an amino acid sequence of RYLLEAKEAENITTG (SEQ ID NO:1), wherein one amino acid residue is optionally replaced with a conservative or nonconservative substitution which is an amino acid or amino acid equivalent having a side chain modification or side chain substitution.

10. The method of claim 9 wherein the amino acid sequence of the peptide consists of RYLLEAKEAENITIG (SEQ ID NO:1).

11. The method of any one of claim 9 or 10, wherein the cancer is infiltrating breast cancer, preinvasive breast cancer, inflammatory breast cancer, Paget's disease, metastatic breast cancer, recurrent breast cancer, appendix cancer, bile duct cancer, extrahepatic bile duct cancer, colon cancer, esophageal cancer, gallbladder cancer, gastric cancer, intestinal cancer, liver cancer, pancreatic cancer, rectal cancer, stomach cancer, adrenal cancer, bladder cancer, kidney cancer, penile cancer, prostate cancer, testicular cancer, urinary cancer, cervical cancer, endrometrial cancer, fallopian tube cancer, ovarian cancer, uterine cancer, vaginal cancer, vulvar cancer, eye cancer, head and neck cancer, jaw cancer, laryngeal cancer, pharyngeal cancer, oral cancer, nasal cavity cancer, salivary gland cancer, sinus cancer, throat cancer, thyroid cancer, tongue cancer, tonsil cancer, Hodgkin's disease, leukemia, acute lymphocytic leukemia, acute granulocytic leukemia, acute myelogenous leukemia, chronic lymphatic leukemia, chronic myelogenous leukemia, multiple myeloma, lymphoma, b-cell lymphoma, lymph node cancer, bone cancer, osteosarcoma, melanoma, skin cancer, basal cell cancer, squamous cell cancer, sarcoma, Ewing's sarcoma, Kaposi's sarcoma, brain cancer, astrocytoma, glioblastoma, glioma, pituitary gland cancer, spinal cord cancer, lung cancer, adenocarcinoma, oat cell cancer, non-small cell lung cancer, small cell lung cancer, squamous cell cancer, or mesothelioma.

12. The methods of claim 6, wherein the disease associated with tissue damage is inflammation.

13. The method of claim 12, wherein the inflammation is appendicitis, blepharitis, bronchitis, bursitis, cervicitis, cholangitis, cholecystitis, chorioamnionitis, conjunctivitis, cystitis, dacryoadenitis, dermatitis, endocarditis, endometritis, ep.icondylitis, epididymitis, fibrositis, gastritis, gingivitis, glossitiss, hidradenitis suppurativa, iritis, laryngitis, mastitis, myocarditis, myositis, nephritis, omphalitis, oophoritis, orchitis, osteitis, otitis, parotitis, pericarditis, peritonitis, pharyngitis, pleuritis, phlebitis, pneumonitis (pneumonia), prostatitis, pyelonephritis, rhinitis, salpingitis, sinusitis, stomatitis, synovitis, tonsillitis, uveitis, urethritis, vaginitis, vulvitis, asthma, systemic lupus erythematosus, myasthenia gravis, tendonitis, angiitis, chronic bronchitis, pancreatitis, osteomyelitis, rheumatoid arthritis, psoriatic arthritis, glumeronephritis, optic neuritis, temporal arteritis, encephalitis, meningitis, traverse myelitis, dermatomyositis, polymyositis, neerotizing fasciitis, hepatitis, neerotizina entereolitis, pelvic inflammatory disease, inflammatory bowel disease, ulcerative colitis, Chron's disease, ileitis, enteritis, proctitis, vasculitis, vascular stenosis, restenosis, hypotension, Type-1 diabetes, Kawasaki disease, Decum's disease, chronic obstructive pulmonary disease, psoriasis, artherosclerosis, scleroderma, Sjogren's syndrome, mixed connective tissue disease, rosacea, gastric ulcers, duodenal ulcers, Alzheimer's disease, adult onset Still's disease, acute retinal pigment epithchitis, Tietze's syndrome, Bechçet's disease, white dot syndrome, acute posterior multifocal placoid pigment epitheliopathy, serpiginous choroiditis, birdshot chorloretinopathy, multifocal choroiditis with panuveitis, diffuse subretinal fibrosis syndrome, punctuate inner choroidopathy, multiple evanescent white dot syndrome, diffuse unilateral subacute neuroretinitis, granuloma annulare, irritable bowel syndrome, gastroenteritis, Grave's disease, multiple sclerosis, Dupuytren's contracture, graft rejection diseases, allograft rejection, graft-v-host disease, skin graft rejection, solid organ transplant rejection, bone marrow transplant rejection, inflammatory dermatoses, viral cutaneous pathologies such as those derived from human papilloma virus, HIV, or RLV infection, bacterial, fungal and or other parasital cutaneous pathologies, cutaneous lupus erythematosus. Hyper IgG4 disease, allergies, allergic diseases, inflammation resulting from blunt trauma, inflammation resulting from contusions, inflammation resulting allergies, rheumatic disease, childhood arthritis, rheumatoid arthritis, Churg-Strauss syndrome, fibromyalgia, giant cell (temporal) arteritis, gout, Henoch-Schoenlin purpura, hypersensitivity vasculitis, ankylosing spondylitis, capsulitis, rheumatic fever, rheumatic heart disease, systemic lupus erythematosus. polymyalgia rheumatica, osteoarthritis, polyarteritis nodosa, Reiter's syndrome, sports related injuries, runner's knee, tennis elbow, frozen shoulder, Achilles tendonitis, plantar fasciitis, bursitis, Osgood-Schlatter disease, repetitive stress injuries, cumulative trauma diseases, focal dystonia, carpal tunnel syndrome, intersection syndrome, reflex sympathetic dystrophy syndrome, stenosing tenosynovitis, De Quervain's syndrome, trigger finger/trigger thumb, thoracic outlet syndrome, tendonitis, tenosynovitis, radial tunnel syndrome, Raynaud's disease, ganglion, gamer's thumb, Wii-itis, inflammation resulting from infections.

14. The methods of claim 6, wherein the disease associated with tissue damage results from exposure to a toxic agent.

15. The method of claim 14, wherein the toxic agent is a biological agent, chemical agent or radiation agent.

16. The method of any one of claims 6-15, wherein the subject is a mammal.

17. The method of claim 16, wherein the mammal is a human.

18. A method for protecting, maintaining, or enhancing the viability of a responsive cell, tissue, or organ isolated from a mammal comprising exposing the cell, tissue, or organ to a pharmaceutical composition of claim 3. 19, An isolated. nucleic acid comprising a nucleotide sequence encoding the isolated peptide of any one of claims 1-2.

20. An expression vector comprising the nucleic acid of claim 19.

21. A host cell comprising the expression vector of claim 20.

22. A method of recombinanty producing an isolated peptide comprising the step of isolating the peptide from the host cell of claim 21.


SEQUENCE LISTING (3 sequences)

SEQ ID NO: 1 - protein, 15 aa, Artificial Sequence
  peptide that have tissue protective activity
RYLLEAKEAENITTG

SEQ ID NO: 2 - protein, 16 aa, Artificial Sequence
  peptide that have tissue protective activity
RYLLEAKEAENITTGC

SEQ ID NO: 3 - protein, 11 aa, Artificial Sequence
  PEG modified peptide that have tissue protective activity
  MOD_RES (1)..(1): PEG modified Gln residue
XEQLERALNSS